SEQUENCE LISTING (43 sequences)

SEQ ID NO: 1 - DNA, 1857 bp, Pycnoporus sanguineus BRFM49
atgtcacacttcatcgttactgggcctgtaggaggtcagactgagggcgctcctgctcccaaccgcctcgaaatcaacgacttcgtcaagaatgaggagttcttctcgctttacgtccaggctctcgatatcatgtatggactgaagcaggaggaactgatctcgttcttccagatcggtggcattcatggattgccatacgttgcctggagtgatgccggagcggatgaccctgctgagccgtccgggtactgtacccatggctccgtactgttcccgacctggcataggccttacgtcgcactatatgagcaaatcttgcacaagtatgctggagagatcgctgataagtacacggtcgacaaaccgcgttggcagaaggcagcggccgacctgcgccaacccttctgggactgggccaagaacacgctgcctcctcctgaagtcatctctctcgacaaagtcacgattacgacaccagatggacagaggacgcaagttgacaatccactccgtcgctaccgcttccatccgatcgaccccagcttcccagagccatacagcaactggccagcgacactgagacatccgacaagtgatggctcggatgccaaagacaacgtgaaggatctcactactactctgaaggcggaccagcctgatatcacgacgaagacgtataatctattgaccagagtgcacacgtggccggcgttcagcaaccacactccaggcgatggcggcagctccagtaacagtcttgaggccattcacgaccacatccatgactcagttggcggcggaggccagatgggagacccgtccgtggcaggcttcgacccaatcttcttcctgcaccattgccaagttgatcgtcttcttgcactgtggtccgccttgaaccccggcgtgtgggtcaacagctctagctccgaagatggcacctacacgatcccgcctgactctaccgtggaccaaactactgcattgacgcccttctgggatacccaaagcacattctggacgtccttccagtctgctggagtctcgcccagccaatttggctattcttaccccgagtttaacggtctcaacctgcaagatcagaaggctgtgaaagatcacatcgccgaggtcgtgaacgagctctacggtcatcgcatgcggaaaaccttccctttcccccagctccaggcagtttccgtagccaagcagggcgacgccgtcactccatccgtggctaccgattcagtgtcgtcttctaccacacctgccgaaaatcccgcatcccgcgaggatgcctctgataaggacacagagccgacgctcaatgtagaggttgccgcgccaggcgcgcacttgacctccaccaagtattgggactggactgctcgcattcatgtcaagaagtacgaagtcggaggcagcttcagcgtcctgctcttcctgggtgcaatccccgagaacccagcggattggcgcacgagccccaactacgttggcggtcatcatgctttcgtgaatagctcaccgcagcgctgcgctaactgccgtggtcaaggcgaccttgtcatcgaaggcttcgtccatctcaacgaggcgatcgcccgccatgcgcacctcgactccttcgatccaaccgtcgtgaggccgtacctcacgcgcgagttgcactggggtgtgatgaaggtgaatggcaccgtcgtgcccctgcaagacgtcccgtcgctcgaggttgtcgtcctctcaactcctcttacccttcctccgggagagccattccctgtccccggaacgcccgtcaatcatcatgacatcacccatggacgtcctggtggctctcaccacacgcactaa

SEQ ID NO: 2 - protein, 618 aa, Pycnoporus sanguineus BRFM49
MSHFIVTGPVGGQTEGAPAPNRLEINDFVKNEEFFSLYVQALDIMYGLKQEELISFFQIGGIHGLPYVAWSDAGADDPAEPSGYCTHGSVLFPTWHRPYVALYEQILHKYAGEIADKYTVDKPRWQKAAADLRQPFWDWAKNTLPPPEVISLDKVTITTPDGQRTQVDNPLRRYRFHPIDPSFPEPYSNWPATLRHPTSDGSDAKDNVKDLTTTLKADQPDITTKTYNLLTRVHTWPAFSNHTPGDGGSSSNSLEAIHDHIHDSVGGGGQMGDPSVAGFDPIFFLHHCQVDRLLALWSALNPGVWVNSSSSEDGTYTIPPDSTVDQTTALTPFWDTQSTFWTSFQSAGVSPSQFGYSYPEFNGLNLQDQKAVKDHIAEVVNELYGHRMRKTFPFPQLQAVSVAKQGDAVTPSVATDSVSSSTTPAENPASREDASDKDTEPTLNVEVAAPGAHLTSTKYWDWTARIHVKKYEVGGSFSVLLFLGAIPENPADWRTSPNYVGGHHAFVNSSPQRCANCRGQGDLVIEGFVHLNEAIARHAHLDSFDPTVVRPYLTRELHWGVMKVNGTVVPLQDVPSLEVVVLSTPLTLPPGEPFPVPGTPVNHHDITHGRPGGSHHTH

SEQ ID NO: 3 - DNA, 1047 bp, Escherichia coli
atgaaggcaatccagtacacgagaatcggcgcggaacccgaactcacggagattcccaaacccgagcccggtccaggtgaagtgctcctggaagtcaccgctgccggcgtctgccactcggacgacttcatcatgagcctgcccgaagagcagtacacctacggccttccgctcacgctcggccacgaaggcgcaggcaaggtcgccgccgtcggcgagggtgtcgaaggtctcgacatcggaaccaatgtcgtcgtctacgggccttggggttgtggcaactgttggcactgctcacaaggactcgagaactattgctctcgcgcccaagaactcggaatcaatcctcccggtctcggtgcacccggcgcgttggccgagttcatgatcgtcgattctcctcgccaccttgtcccgatcggtgacctcgacccggtcaagacggtgccgctgaccgacgccggtctgacgccgtatcacgcgatcaagcgttctctgccgaaacttcgcggaggctcgtacgcggttgtcattggtaccggcgggctcggccacgtcgccattcagctcctccgtcacctctcggcggcaacggtcatcgctttggacgtgagcgcggacaagctcgaactggcaaccaaggtaggcgctcacgaagtggttctgtccgacaaggacgcggccgagaacgtccgcaagatcactggaagtcaaggcgccgcactggttctcgacttcgtcggctaccagcccaccatcgacaccgcgatggctgtcgccggcgtcggatcagacgtcacgatcgtcgggatcggggacggccaggcccacgccaaagtcgggttcttccaaagtccttacgaggcttcggtgacagttccgtattggggtgcccgcaacgagttgatcgaattgatcgacctcgcccacgccggcatcttcgacatcgcggtggagaccttcagtctcgacaacggtgccgaagcgtatcgacgactggctgccggaacgctaagcggccgtgcggttgtggtccctggtctgtag

SEQ ID NO: 4 - protein, 348 aa, Escherichia coli
MKAIQYTRIGAEPELTEIPKPEPGPGEVLLEVTAAGVCHSDDFIMSLPEEQYTYGLPLTLGHEGAGKVAAVGEGVEGLDIGTNVVVYGPWGCGNCWHCSQGLENYCSRAQELGINPPGLGAPGALAEFMIVDSPRHLVPIGDLDPVKTVPLTDAGLTPYHAIKRSLPKLRGGSYAVVIGTGGLGHVAIQLLRHLSAATVIALDVSADKLELATKVGAHEVVLSDKDAAENVRKITGSQGAALVLDFVGYQPTIDTAMAVAGVGSDVTIVGIGDGQAHAKVGFFQSPYEASVTVPYWGARNELIELIDLAHAGIFDIAVETFSLDNGAEAYRRLAAGTLSGRAVVVPGL

SEQ ID NO: 5 - DNA, 1563 bp, Escherichia coli
atgaaaccagaagatttccgcgccagtacccaacgtccgttcaccggggaagagtatctgaaaagcctgcaggatggtcgcgagatctatatctatggcgagcgagtgaaagacgtcactactcatccggcatttcgtaatgcggctgcgtctgttgcccaactgtacgacgcgctacacaaaccggagatgcaggactctctgtgctggaacaccgacaccggcagcggcggctatacccataaattcttccgcgtggcgaaaagtgccgacgacctgcgccacgaacgcgatgccatcgctgagtggtcacgcctgagctatggctggatgggccgtaccccagactacaaagctgctttcggttgcgcactgggcggaactccgggcttttacggtcagttcgagcagaacgcccgtaactggtacacccgtattcaggaaactggcctctactttaaccacgcgattgttaacccaccgatcgatcgtcatttgccgaccgataaagtaaaagacgtttacatcaagctggaaaaagagactgacgccgggattatcgtcagcggtgcgaaagtggttgccaccaactcggcgctgactcactacaacatgattggcttcggctcggcacaagtaatgggcgaaaacccggacttcgcactgatgttcgttgcgccaatggatgccgatggcgtcaaattaatctcccgcgcctcttatgagatggtcgcgggtgctaccggctcaccgtatgactacccgctctccagccgcttcgatgagaacgatgcgattctggtgatggataacgtgctgatcccatgggaaaacgtgctgctctaccgcgattttgatcgctgccgtcgctggacgatggaaggcggtttcgcccgtatgtatccgctgcaagcctgtgtgcgcctggcagtgaaactcgacttcattacggcactgctgaaaaaatcactcgaatgtaccggcaccctggagttccgtggtgtgcaggccgatctcggtgaagtggtggcgtggcgcaacaccttctgggcattgagtgactcgatgtgttctgaagcgacgccgtgggtcaacggggcttatttaccggatcatgccgcactgcaaacctatcgcgtactggcaccaatggcctacgcgaagatcaaaaacattatcgaacgcaacgttaccagtggcctgatctatctcccttccagtgcccgtgacctgaacaatccgcagatcgaccagtatctggcgaagtatgtgcgcggttcgaacggtatggatcacgtccagcgcatcaagatcctcaaactgatgtgggatgccattggcagcgagtttggtggtcgtcacgaactgtatgaaatcaactactccggtagccaggatgagattcgcctgcagtgtctgcgccaggcacaaagctccggcaatatggacaagatgatggcgatggttgatcgctgcctgtcggaatacgaccagaacggctggactgtgccgcacctgcacaacaacgacgatatcaacatgctggataagctgctgaaataa

SEQ ID NO: 6 - protein, 520 aa, Escherichia coli
MKPEDFRASTQRPFTGEEYLKSLQDGREIYIYGERVKDVTTHPAFRNAAASVAQLYDALHKPEMQDSLCWNTDTGSGGYTHKFFRVAKSADDLRHERDAIAEWSRLSYGWMGRTPDYKAAFGCALGGTPGFYGQFEQNARNWYTRIQETGLYFNHAIVNPPIDRHLPTDKVKDVYIKLEKETDAGIIVSGAKVVATNSALTHYNMIGFGSAQVMGENPDFALMFVAPMDADGVKLISRASYEMVAGATGSPYDYPLSSRFDENDAILVMDNVLIPWENVLLYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQSSGNMDKMMAMVDRCLSEYDQNGWTVPHLHNNDDINMLDKLLK

SEQ ID NO: 7 - DNA, 513 bp, Escherichia coli
atgcaattagatgaacaacgcctgcgctttcgtgacgcaatggccagcctgtcggcagcggtaaatattatcaccaccgagggcgacgccggacaatgcgggattacggcaacggccgtctgctcggtcacggatacaccaccatcgctgatggtgtgcattaacgccaacagtgcgatgaacccggtttttcagggcaacggtaagttgtgcgtcaacgtcctcaaccatgagcaggaactgatggcacgccacttcgcgggcatgacaggcatggcgatggaagagcgttttagcctctcatgctggcaaaaaggtccgctggcgcagccggtgctaaaaggttcgctggccagtcttgaaggtgagatccgcgatgtgcaggcaattggcacacatctggtgtatctggtggagattaaaaacatcatcctcagtgcagaaggtcacggacttatctactttaaacgccgtttccatccggtgatgctggaaatggaagctgcgatttaa

SEQ ID NO: 8 - protein, 170 aa, Escherichia coli
MQLDEQRLRFRDAMASLSAAVNIITTEGDAGQCGITATAVCSVTDTPPSLMVCINANSAMNPVFQGNGKLCVNVLNHEQELMARHFAGMTGMAMEERFSLSCWQKGPLAQPVLKGSLASLEGEIRDVQAIGTHLVYLVEIKNIILSAEGHGLIYFKRRFHPVMLEMEAAI

SEQ ID NO: 9 - DNA, 1413 bp, Escherichia coli
gtgacccccgaacaattccgccagtacggccaccaactgatcgacctgattgccgactaccgccagaccgtgggcgaacgcccggtcatggcccaggtcgaacctggctatctcaaggccgccttgcccgcaactgcccctcaacaaggcgaacctttcgcggccattctcgacgacgtcaataacctggtcatgcccggcctgtcccattggcagcacccggacttctatggctatttcccttccaatggcaccctgtcctcggtgctgggggacttcctcagtaccggtctgggcgtgctgggcctgtcctggcaatccagcccggccctgagcgaactggaagaaaccaccctcgactggctgcgccagttgcttggcctgtctggccagtggagtggggtgatccaggacactgcctcgaccagcaccctggtggcgctgatcagtgcccgtgaacgcgccactgactacgccctggtacgtggtggcctgcaggccgagcccaagcctttgatcgtgtatgtcagcgcccacgcccacagctcggtggacaaggctgcactgctggcaggttttggccgcgacaatatccgcctgattcccaccgacgaacgctacgccctgcgcccagaggcactgcaggcggcgatcgaacaggacctggctgccggcaaccagccgtgcgccgtggttgccaccaccggcaccacgacgaccactgccctcgacccgctgcgcccggtcggtgaaatcgcccaggccaatgggctgtggttgcacgttgactcggccatggccggttcggcgatgatcctgcccgagtgccgctggatgtgggacggcatcgagctggccgattcggtggtggtcaacgcgcacaaatggctgggtgtggccttcgattgctcgatctactacgtgcgcgatccgcaacacctgatccgggtgatgagcaccaatcccagctacctgcagtcggcggtggatggcgaggtgaagaacctgcgcgactgggggataccgctgggccgtcggttccgtgcgttgaagctgtggttcatgttgcgcagcgagggtgtcgacgcattgcaggcgcggctgcggcgtgacctggacaatgcccagtggctggcggggcaggtcgaggcggcggcggagtgggaagtgttggcgccagtacagctgcaaaccttgtgcattcgccatcgaccggcggggcttgaaggggaggcgctggatgcgcataccaagggctgggccgagcggctgaatgcatccggcgctgcttatgtgacgccggctacactggacgggcggtggatggtgcgggtttcgattggtgcgctgccgaccgagcggggggatgtgcagcggctgtgggcacgtctgcaggacgtgatcaagggctga

SEQ ID NO: 10 - protein, 470 aa, Escherichia coli
MTPEQFRQYGHQLIDLIADYRQTVGERPVMAQVEPGYLKAALPATAPQQGEPFAAILDDVNNLVMPGLSHWQHPDFYGYFPSNGTLSSVLGDFLSTGLGVLGLSWQSSPALSELEETTLDWLRQLLGLSGQWSGVIQDTASTSTLVALISARERATDYALVRGGLQAEPKPLIVYVSAHAHSSVDKAALLAGFGRDNIRLIPTDERYALRPEALQAAIEQDLAAGNQPCAVVATTGTTTTTALDPLRPVGEIAQANGLWLHVDSAMAGSAMILPECRWMWDGIELADSVVVNAHKWLGVAFDCSIYYVRDPQHLIRVMSTNPSYLQSAVDGEVKNLRDWGIPLGRRFRALKLWFMLRSEGVDALQARLRRDLDNAQWLAGQVEAAAEWEVLAPVQLQTLCIRHRPAGLEGEALDAHTKGWAERLNASGAAYVTPATLDGRWMVRVSIGALPTERGDVQRLWARLQDVIKG

SEQ ID NO: 11 - DNA, 2274 bp, Escherichia coli
atgggaagcccctctctgtattctgcccgtaaaacaaccctggcgttggcagtcgccttaagtttcgcctggcaagcgccggtatttgcccacggtggtgaagcgcatatggtgccaatggataaaacgcttaaagaatttggtgccgatgtgcagtgggacgactacgcccagctctttaccctgattaaagatggcgcgtacgtgaaagtgaagcctggtgcgcaaacagcaattgttaatggtcagcctctggcactgcaagtaccggtagtgatgaaagacaataaagcctgggtttctgacacctttattaacgatgttttccagtccgggctggatcaaacctttcaggtagaaaagcgccctcacccacttaatgcgctaactgcggacgaaattaaacaggccgttgaaattgttaaagcttccgcggacttcaaacccaatacccgttttactgagatctccctgctaccgccagataaagaagctgtctgggcgtttgcgctggaaaacaaaccggttgaccagccgcgcaaagccgacgtcattatgctcgacggcaaacatatcatcgaagcggtggtggatctgcaaaacaacaaactgctctcctggcaacccattaaagacgcccacggtatggtgttgctggatgatttcgccagtgtgcagaacattattaacaacagtgaagaatttgccgctgccgtgaagaaacgcggtattactgatgcgaaaaaagtgattaccacgccgctgaccgtaggttatttcgatggtaaagatggcctgaaacaagatgcccggttgctcaaagtcatcagctatcttgatgtcggtgatggcaactactgggcacatcccatcgaaaacctggtggcggtcgttgatttagaacagaaaaaaatcgttaagattgaagaaggtccggtagttccggtgccaatgaccgcacgcccatttgatggccgtgaccgcgttgctccggcagttaagcctatgcaaatcattgagcctgaaggtaaaaattacaccattactggcgatatgattcactggcggaactgggattttcacctcagcatgaactctcgcgtcgggccgatgatctccaccgtgacttataacgacaatggcaccaaacgcaaagtcatgtacgaaggttctctcggcggcatgattgtgccttacggtgatcctgatattggctggtactttaaagcgtatctggactctggtgactacggtatgggcacgctaacctcaccaattgctcgtggtaaagatgccccgtctaacgcagtgctccttaatgaaaccatcgccgactacactggcgtgccgatggagatccctcgcgctatcgcggtatttgaacgttatgccgggccggagtataagcatcaggaaatgggccagcccaacgtcagtaccgaacgccgggagttagtggtgcgctggatcagtacagtgggtaactatgactacatttttgactggatcttccatgaaaacggcactattggcatcgatgccggtgctacgggcatcgaagcggtgaaaggtgttaaagcgaaaaccatgcacgatgagacggcgaaagatgacacgcgctacggcacgcttatcgatcacaatatcgtgggtactacacaccaacatatttataatttccgcctcgatctggatgtagatggcgagaataacagcctggtggcgatggacccagtggtaaaaccgaatactgccggtggcccacgcaccagtaccatgcaagttaatcagtacaacatcggcaatgaacaggatgccgcacagaaatttgatccgggcacgattcgtctgttgagtaacccgaacaaagagaaccgcatgggcaatccggtttcctatcaaattattccttatgcaggtggtactcacccggtagcaaaaggtgcccagttcgcgccggacgagtggatctatcatcgtttaagctttatggacaagcagctctgggtaacgcgttatcatcctggcgagcgtttcccggaaggcaaatatccgaaccgttctactcatgacaccggtcttggacaatacagtaaggataacgagtcgctggacaacaccgacgccgttgtctggatgaccaccggcaccacacatgtggcccgcgccgaagagtggccgattatgccgaccgaatgggtacatactctgctgaaaccatggaacttctttgacgaaacgccaacgctaggggcgctgaagaaagataagtga

SEQ ID NO: 12 - protein, 757 aa, Escherichia coli
MGSPSLYSARKTTLALAVALSFAWQAPVFAHGGEAHMVPMDKTLKEFGADVQWDDYAQLFTLIKDGAYVKVKPGAQTAIVNGQPLALQVPVVMKDNKAWVSDTFINDVFQSGLDQTFQVEKRPHPLNALTADEIKQAVEIVKASADFKPNTRFTEISLLPPDKEAVWAFALENKPVDQPRKADVIMLDGKHIIEAVVDLQNNKLLSWQPIKDAHGMVLLDDFASVQNIINNSEEFAAAVKKRGITDAKKVITTPLTVGYFDGKDGLKQDARLLKVISYLDVGDGNYWAHPIENLVAVVDLEQKKIVKIEEGPVVPVPMTARPFDGRDRVAPAVKPMQIIEPEGKNYTITGDMIHWRNWDFHLSMNSRVGPMISTVTYNDNGTKRKVMYEGSLGGMIVPYGDPDIGWYFKAYLDSGDYGMGTLTSPIARGKDAPSNAVLLNETIADYTGVPMEIPRAIAVFERYAGPEYKHQEMGQPNVSTERRELVVRWISTVGNYDYIFDWIFHENGTIGIDAGATGIEAVKGVKAKTMHDETAKDDTRYGTLIDHNIVGTTHQHIYNFRLDLDVDGENNSLVAMDPVVKPNTAGGPRTSTMQVNQYNIGNEQDAAQKFDPGTIRLLSNPNKENRMGNPVSYQIIPYAGGTHPVAKGAQFAPDEWIYHRLSFMDKQLWVTRYHPGERFPEGKYPNRSTHDTGLGQYSKDNESLDNTDAVVWMTTGTTHVARAEEWPIMPTEWVHTLLKPWNFFDETPTLGALKKDK

SEQ ID NO: 13 - DNA, 1191 bp, Escherichia coli
atgcgcaacatgcaggaaaaaggcgtgtctgaaaaagaaatcctggaagaactgaagaaataccgttccctggatctgaagtatgaagacggtaacatttttggtagcatgtgctccaatgtactgccgattacccgcaaaattgtcgatatttttctggagactaacctgggtgatccaggcctgtttaagggcaccaaactgctggaagaaaaggccgtagctctgctgggctctctgctgaacaacaaagacgcatacggtcacattgtgtctggtggcaccgaagccaacctgatggcgctgcgttgcattaaaaacatctggcgtgaaaaacgtcgcaagggtctgtccaaaaacgagcacccgaaaattatcgttccaattactgctcacttctcctttgaaaaaggtcgcgaaatgatggacctggaatatatctacgctcctatcaaagaagattacactatcgacgagaagttcgtgaaggatgctgtggaagactacgacgtggacggtattatcggcatcgcgggtactaccgaactgggtacgatcgacaacattgaggagctgtctaaaatcgcgaaggaaaacaatatctacatccacgtggacgcagcgttcggtggtctggttatcccatttctggatgacaaatacaaaaagaagggtgttaactacaaattcgacttcagcctgggcgtagacagcattaccatcgatcctcacaagatgggccattgcccaattccgagcggcggtatcctgttcaaagacatcggttacaaacgttacctggacgtggacgctccgtacctgactgaaactcgtcaggcgacgatcctgggcactcgtgtgggctttggcggtgcgtgtacctatgctgtgctgcgttatctgggtcgtgagggtcagcgtaagatcgtgaacgaatgcatggaaaacaccctgtacctgtacaaaaagctgaaagaaaacaacttcaaaccggttatcgagccgatcctgaacattgtggccatcgaagacgaagattacaaagaagtttgtaagaagctgcgtgatcgcggtatctacgtgtctgtgtgtaactgcgttaaggccctgcgtatcgtggtaatgccgcacatcaaacgcgaacacatcgataacttcatcgagattctgaactctatcaaacgcgattaa

SEQ ID NO: 14 - protein, 396 aa, Escherichia coli
MRNMQEKGVSEKEILEELKKYRSLDLKYEDGNIFGSMCSNVLPITRKIVDIFLETNLGDPGLFKGTKLLEEKAVALLGSLLNNKDAYGHIVSGGTEANLMALRCIKNIWREKRRKGLSKNEHPKIIVPITAHFSFEKGREMMDLEYIYAPIKEDYTIDEKFVKDAVEDYDVDGIIGIAGTTELGTIDNIEELSKIAKENNIYIHVDAAFGGLVIPFLDDKYKKKGVNYKFDFSLGVDSITIDPHKMGHCPIPSGGILFKDIGYKRYLDVDAPYLTETRQATILGTRVGFGGACTYAVLRYLGREGQRKIVNECMENTLYLYKKLKENNFKPVIEPILNIVAIEDEDYKEVCKKLRDRGIYVSVCNCVKALRIVVMPHIKREHIDNFIEILNSIKRD

SEQ ID NO: 15 - DNA, 3908 bp, Escherichia coli
gatcatcagtgcagcaaccaccaacatcatcaagaaaccaaatgcttcacttggcatcatgtaattgatcaccacaaccaatgcggtcactgctgatgagatcagcacagcattcattggaataccacgcgtattcactttggttaagaatttgggtgcattgccctgttctgccaaaccatgcagcattcgtgtattacaatatacacagctgttatagaccgacactgccgcaatcaacaccacaaagttcaatacgttggcaacgccattactatcaagcgaatggaaaatcatgacaaatggactaccgccttctgcaacttgattccaaggatataaactgagtaggatcgtaatcgcaccgatatagaaaattaaaacacgataaacaatttgattggtggctttcggaattgatttcttcggatctttggtttcagccgcgctaatcccaattaactctaacccaccaaaggcaaacatgattgcagccatcgccatcataaacccttgtgcaccattcgggaaaaagccaccgagttgccataggttcgatacactcgcttggggacctgctgttccgctaaatagcaaataagcaccaaaagcaatcatactcaaaatggcaaaaatcttaatcaaggaaaggacaaactccgtttcaccaaagaaacgtacgttgatcaagttaatcccgttaattaagacaaagaaaaataatgcagatgcccacgtcggtaactctcgccaccaaaattgcatgaaggttccaatggcactcagttccgccatgcccaccaacacataaagcacccagtaattccagccagacatgaagcctgccattctgccccaatacttatgtgcaaaatggctaaatgaaccactcacaggctcttcaacgaccatttcaccaagttggcgcataattaaaaatgcaatgacacctgccaaggcataaccgagaatcactgaaggaccagccaatttaatggtttgtgaaagccctagaaataatcctgtgccaattgcaccacccagtgcaatcagctggatatgacgattcgtcaaatccttctttaaaccgttagatttctcaatttccataattttccctaacttgctataaattccattacagcattttatcattcatataaacagaactttaaagccttgttcctgtttttatctcgcttgcatgtgcttcctttactcaggttagttatgcttaaaagattattcatcactacagcacaaaaccgacagcatccatttcatctatacaaatcaattatttatttgattctacttaggatggagtttgacttatacgttttgcattgaatcatgactcaacacaaaagatcgtcatgtatgcgcgatcagcttattttcaaccctatgcaaatctagctaaccaagttttctataccgatttaatttccaaacatcctctgtttggcacacttgaatcctaactgctgacgcttaaaaatacaatataattccatgtatttctacatcttaattaaaaacaaatacacttcgaattgaagaaagaattgtaatttacttctcaatgctaatctaaattaagtgtttttaatgcattatttgggccgataatcacacgacttcatcccagtgatggcagataaaataagactgacgatttcgatatctcaaaattacggttcaagaaaaaaacaaaaaaactttttacttgtaatagatgatatctaaatcgcgcgtcttaaatcatgttgtttactaaacaaccaactagaatgcaatcgcctttttatattcggtatcttgctgaggatgtttcagtttttgataggtcatatcaccataatcataacgtgcaaccaaatctgcaaatttggttaaattacttggtgcatccattgcagctagctttaactcgaccacggcaagatttggatgctcagaaagttgcgctaaagtgtctttgagctcagctaaattttcgaccataaatgtatcgtattgaccttgaccattaaagaccttgaccatctcggtatatttccagttttgaacatcgttatatttcgcgttctcacctaaaatcagacgttcaatggtatagccgccattgtttaaaataaaaataatcggctttaggtcttcgcgaataatagttgacaactcttgcatggtcagctgaatcgaaccatccccaataaacagcacatgacgtcgtttgggtgcagcgaccatactgccgagcaatgctggtaaggtataaccaattgatccccaaagtggttgtgagatatagcgggcttgtttcggtaaacgcatactcgataacgcagaatttgacgtgcctacctcaccaataatcacatcatcatcacgcaagaactgccccacttcttgccacaattgcaagtgtgtcaatggacgttttaactcttcttctgcaggtacaggagtactcgcggccagtggtgccgctaaagtcggtttagagactttacgcacagcaacctgatcaagcaaattgcttaacagctcttgaatttcaatcccagggtagttttcttgatcgatcgtgacatcgtattgtttaatctcaatataatgatctgtattaatacgatgagtaaagtaagcggaaccaacatcactaaaacgcacaccaatcccaatcaagcaatctgactgttcaatcaactttctggttgctgcaggccccacagcaccgacatatacgccggcatataatggagaggattcatccatggtgtttttggtggtatttaaacacgcataaggaatgccacatttttctgcaagttgtcccagcaatgtcgtcacttggaaggtatgtgcatcatgatcaatcagtaatgcaggattcttggcttggctaatctgttcgctgagtaactgcacaacatgtgcaagcacctctggatcacttttcggtttagacaaatctagtgtacgaccatcgacgtcgattttgacatgcgtaatatcagaaggaagttggatatagactggacgacgttcaatccaacattgacgcaatacccggtcaatttcagcagcagcatttgcaggagtaatacgcgtttgagcaacactgaactctttcatacaatttaaaatattttgataattgccatcaaccaaggtgtgatgcagtaatgcgccctgttctacagcgtgtaatggcggtataccagagatcaccacaacaggtactttttctgcatacgcgcccgcaacgccattgatagcactgagatcacctacaccataggtggtgagtaaagcaccaaaaccattgatacgcgcataaccatctgcggcataggctgcatttaattcattacaattgccaataaatgccaatttagcatctgcttcaacttgctctaaataacttaaattaaagtcacctggcacaccaaaaagatgctgtacaccaagctcagccagacgctgatttaaataacaaccaatctcaataaacatttctacttccctgcaaaataattgttgttataaacagaataggtcaattcattttgtatattcgtgcataatagagtcataaatttaaaaaaatgcacagaatgtgtatcgcaaaggaatttcatgcaatggataagtttgattggcaaatcattcatgcgttacaacgcaacggtaggctcaccaatcaagaaattggcgatttgattggcctttctgcctctcaatgttcccgcagaagacaagttcttgaacaaaaaagtattattttaggctatagcgcaagaataaatccaaatgcgcttggaatttcaattaccaccatgattcatgtcaacctcaagaaccacggcgccaatcccaaacatgccatacatgatctgatc

SEQ ID NO: 16 - DNA, 1731 bp, Escherichia coli
atgtttattgagattggttgttatttaaatcagcgtctggctgagcttggtgtacagcatctttttggtgtgccaggtgactttaatttaagttatttagagcaagttgaagcagatgctaaattggcatttattggcaattgtaatgaattaaatgcagcctatgccgcagatggttatgcgcgtatcaatggttttggtgctttactcaccacctatggtgtaggtgatctcagtgctatcaatggcgttgcgggcgcgtatgcagaaaaagtacctgttgtggtgatctctggtataccgccattacacgctgtagaacagggcgcattactgcatcacaccttggttgatggcaattatcaaaatattttaaattgtatgaaagagttcagtgttgctcaaacgcgtattactcctgcaaatgctgctgctgaaattgaccgggtattgcgtcaatgttggattgaacgtcgtccagtctatatccaacttccttctgatattacgcatgtcaaaatcgacgtcgatggtcgtacactagatttgtctaaaccgaaaagtgatccagaggtgcttgcacatgttgtgcagttactcagcgaacagattagccaagccaagaatcctgcattactgattgatcatgatgcacataccttccaagtgacgacattgctgggacaacttgcagaaaaatgtggcattccttatgcgtgtttaaataccaccaaaaacaccatggatgaatcctctccattatatgccggcgtatatgtcggtgctgtggggcctgcagcaaccagaaagttgattgaacagtcagattgcttgattgggattggtgtgcgttttagtgatgttggttccgcttactttactcatcgtattaatacagatcattatattgagattaaacaatacgatgtcacgatcgatcaagaaaactaccctgggattgaaattcaagagctgttaagcaatttgcttgatcaggttgctgtgcgtaaagtctctaaaccgactttagcggcaccactggccgcgagtactcctgtacctgcagaagaagagttaaaacgtccattgacacacttgcaattgtggcaagaagtggggcagttcttgcgtgatgatgatgtgattattggtgaggtaggcacgtcaaattctgcgttatcgagtatgcgtttaccgaaacaagcccgctatatctcacaaccactttggggatcaattggttataccttaccagcattgctcggcagtatggtcgctgcacccaaacgacgtcatgtgctgtttattggggatggttcgattcagctgaccatgcaagagttgtcaactattattcgcgaagacctaaagccgattatttttattttaaacaatggcggctataccattgaacgtctgattttaggtgagaacgcgaaatataacgatgttcaaaactggaaatataccgagatggtcaaggtctttaatggtcaaggtcaatacgatacatttatggtcgaaaatttagctgagctcaaagacactttagcgcaactttctgagcatccaaatcttgccgtggtcgagttaaagctagctgcaatggatgcaccaagtaatttaaccaaatttgcagatttggttgcacgttatgattatggtgatatgacctatcaaaaactgaaacatcctcagcaagataccgaatataaaaaggcgattgcattc

SEQ ID NO: 17 - protein, 577 aa, Escherichia coli
MFIEIGCYLNQRLAELGVQHLFGVPGDFNLSYLEQVEADAKLAFIGNCNELNAAYAADGYARINGFGALLTTYGVGDLSAINGVAGAYAEKVPVVVISGIPPLHAVEQGALLHHTLVDGNYQNILNCMKEFSVAQTRITPANAAAEIDRVLRQCWIERRPVYIQLPSDITHVKIDVDGRTLDLSKPKSDPEVLAHVVQLLSEQISQAKNPALLIDHDAHTFQVTTLLGQLAEKCGIPYACLNTTKNTMDESSPLYAGVYVGAVGPAATRKLIEQSDCLIGIGVRFSDVGSAYFTHRINTDHYIEIKQYDVTIDQENYPGIEIQELLSNLLDQVAVRKVSKPTLAAPLAASTPVPAEEELKRPLTHLQLWQEVGQFLRDDDVIIGEVGTSNSALSSMRLPKQARYISQPLWGSIGYTLPALLGSMVAAPKRRHVLFIGDGSIQLTMQELSTIIREDLKPIIFILNNGGYTIERLILGENAKYNDVQNWKYTEMVKVFNGQGQYDTFMVENLAELKDTLAQLSEHPNLAVVELKLAAMDAPSNLTKFADLVARYDYGDMTYQKLKHPQQDTEYKKAIAF

SEQ ID NO: 18 - DNA, 1506 bp, Ralstonia pickettii
atggcgttactggaacgcgccgcgtggtacgacatcgcacgcacgaccaactggaccccgagctacgtcaccgagtccgagctgtttcccgacatcatgaccggcgcgcagggcgtaccgatggagacctgggaaacctacgacgaaccctacaagacgtcgtatcccgaatacgtcagcattcaacgcgagaaggatgccggagcgtactcggtcaaggccgcgctggagcgcagccgcatgttcgaagacgccgacccgggctggctgtcgatcctgaaggcgcactacggcgccattgcgctcggcgaatacgcagcgatgagcgccgaggcacgcatggcccgcttcggccgcgcgccgggcatgcgcaacatggccaccttcggcatgctcgatgagaaccggcacggccagctgcagttgtatttcccgcacgactattgcgccaaggaccgtcagttcgattgggcccataaggcttatcacaccaacgaatggggcgcgatcgcggcacgcagcacgttcgacgatctgttcatgtcgcgcagcgcgatcgacattgcgatcatgctcacgttcgcgttcgagacgggctttaccaacatgcagttcctcggtctcgcggccgacgctgcagaggcgggggatttcacctttgccagcctgatctcaagcatccagaccgacgagtcgcggcatgcacagatcggtggtccggctctgcagatcctgatcgcaagcggccgcaaggaacaggcgcagaaactcgtcgacatcgccattgcgcgggcctggcggctgttctcgctgctcaccggcacctcgatggattacgcaacgccgctgcaccatcgcaaggagtcgttcaaggagttcatgactgagtggatcgtcgggcagtttgaacgcaccttgatcgacctgggcctggacctgccctggtactgggatcagatgatcaacgagttcgactaccagcatcacgcctatcagatgggcatctggttctggcgcccgacgatctggtggaaccccgctgccggcatcacgcccgattgccgcgactggctcgaagagaaataccccggctggaacgacacgttcggcaaggcctgggacgtcatcatcgacaacctgctggccggcaagcccgagctgaccgtgcccgagacactgcccatcgtctgcaacatgagccagttgccgatctgcgcggttccgggtaacggctggatcgtgaaggactacccgctcgactacaagggccgcacgtaccacttcaattccgagatcgaccgctgggtcttccagcaggacccgctgcgctatcgcgaccacctgacgctggtcgaccgattcctcgccggccagatccagccgcccaacctgatgggcgcgcttcagtacatgaacctggcgcctggcgagtgcggcgacgacgcccatcactacgcgtgggtcgaggcgtaccgcaatcagcgctaccagaagaaagccgcttga

SEQ ID NO: 19 - protein, 501 aa, Ralstonia pickettii
MALLERAAWYDIARTTNWTPSYVTESELFPDIMTGAQGVPMETWETYDEPYKTSYPEYVSIQREKDAGAYSVKAALERSRMFEDADPGWLSILKAHYGAIALGEYAAMSAEARMARFGRAPGMRNMATFGMLDENRHGQLQLYFPHDYCAKDRQFDWAHKAYHTNEWGAIAARSTFDDLFMSRSAIDIAIMLTFAFETGFTNMQFLGLAADAAEAGDFTFASLISSIQTDESRHAQIGGPALQILIASGRKEQAQKLVDIAIARAWRLFSLLTGTSMDYATPLHHRKESFKEFMTEWIVGQFERTLIDLGLDLPWYWDQMINEFDYQHHAYQMGIWFWRPTIWWNPAAGITPDCRDWLEEKYPGWNDTFGKAWDVIIDNLLAGKPELTVPETLPIVCNMSQLPICAVPGNGWIVKDYPLDYKGRTYHFNSEIDRWVFQQDPLRYRDHLTLVDRFLAGQIQPPNLMGALQYMNLAPGECGDDAHHYAWVEAYRNQRYQKKAA

SEQ ID NO: 20 - DNA, 990 bp, Ralstonia pickettii
atgacaacgcaagctgaagtcctcaagccgctcaagacctggagccatctggccgcgcggcgacgcaagcccagcgagtacgaaatcgtctcgaccaacctgcactacaccaccgacaacccggatgcgccgttcgaactcgacccgaatttcgagatggcgcagtggttcaagcgcaaccgcaacgcatcgcccctgacccaccccgactggaacgcgttccgcgatccggatgaactggtctaccgcacgtacaacatgctgcaggacgggcaggagacctatgtgttcgggctgctcgaccagttttccgagcgcgggcacgacgccatgctcgaacgcacctgggccggcacgctggcacgcctgtacacgcccgtgcgctacctgttccacacgctgcagatgggctcggcctatctgacgcaactggcgcccgcctcgaccatctcgaactgcgcggcgtaccagacggccgattcgctgcgctggctgacacacaccgcttaccgcaccaaggagctgtcgcagaccttcagcgacctcggcttcggcaccgatgaacgccgctactgggagcaggacccggcctggcaaggctggcgcaagctggtcgaacacgcgctggtggcgtgggactgggccgagtgcttcgttgccctgagcctggtggtgcggccggcagtggaggaagccgtcttgcgcagcctcggcgaagccgcccggcataacggcgacaccttgctgggcctgctgaccgacgcgcaactcgccgatgcgcaacgccatcggcgctgggccggcgcattggtgcgcatggcgctggagcaacccggaaaccgcgaagtcatcaccggttggctcgccaagtgggagcccctggcggatgaagccatcgtggcctactgctcggccctgcccgaggcgcctgcggcccaggcacgcgcaaccgctgcggtgcgcgagttccggcacagcctcggcctgtga

SEQ ID NO: 21 - protein, 329 aa, Ralstonia pickettii
MTTQAEVLKPLKTWSHLAARRRKPSEYEIVSTNLHYTTDNPDAPFELDPNFEMAQWFKRNRNASPLTHPDWNAFRDPDELVYRTYNMLQDGQETYVFGLLDQFSERGHDAMLERTWAGTLARLYTPVRYLFHTLQMGSAYLTQLAPASTISNCAAYQTADSLRWLTHTAYRTKELSQTFSDLGFGTDERRYWEQDPAWQGWRKLVEHALVAWDWAECFVALSLVVRPAVEEAVLRSLGEAARHNGDTLLGLLTDAQLADAQRHRRWAGALVRMALEQPGNREVITGWLAKWEPLADEAIVAYCSALPEAPAAQARATAAVREFRHSLGL

SEQ ID NO: 22 - DNA, 261 bp, Ralstonia pickettii
atggcactttttcctgtgatttccaactttcagtacgacttcgtgctgcaactcgtcgcggtggatacggaaaacaccatcgacgaggtggccgcagcagcggcacaccactcggtgggacgccgcgtggcaccgcagcccggcaagatcgtcagggtgcggcgccagggcggcgagcagttctacccgcgtaacgccaggctggccgacaccgacatcaagccgatggaagcgctcgaattcattttttgcgatgcatga

SEQ ID NO: 23 - protein, 86 aa, Ralstonia pickettii
MALFPVISNFQYDFVLQLVAVDTENTIDEVAAAAAHHSVGRRVAPQPGKIVRVRRQGGEQFYPRNARLADTDIKPMEALEFIFCDA

SEQ ID NO: 24 - DNA, 1503 bp, Pseudomonas mendocina KR1
atggcgatgcacccacgtaaagactggtatgaactgaccagggcgacaaattggacacctagctatgttaccgaagagcagcttttcccagagcggatgtccggtcatatgggtatcccgctggaaaaatgggaaagctatgatgagccctataagacatcctatccggagtacgtaagtatccaacgtgaaaaggatgcaggtgcttattcggtgaaggcggcacttgagcgtgcaaaaatttatgagaactctgacccaggttggatcagcactttgaaatcccattacggcgccatcgcagttggtgaatatgcagccgtaaccggtgaaggtcgtatggcccgtttttcaaaagcaccgggaaatcgcaacatggctacgtttggcatgatggatgaactgcgccatggccagttacagctgtttttcccgcatgaatactgtaagaaggatcgccagtttgattgggcatggcgggcctatcacagtaacgaatgggcagccattgctgcaaagcatttctttgatgacatcattaccggacgtgatgcgatcagcgttgcgatcatgttgacgttttcattcgaaaccggcttcaccaacatgcagtttcttgggttggcggcagatgccgcagaagcaggtgactacacgtttgcaaacctgatctccagcattcaaaccgatgagtcgcgtcatgcacaacagggcggccccgcattacagttgctgatcgaaaacggaaaaagagaagaagcccaaaagaaagtcgacatggcaatttggcgtgcctggcgtctatttgcggtactaaccgggccggttatggattactacacgccgttggaggaccgcagccagtcattcaaggagtttatgtacgagtggatcatcggacagttcgaacgctcgttgatagatctgggcttggacaagccctggtactgggatctattcctcaaggatattgatgagcttcaccatagttatcacatgggtgtttggtactggcgtacaaccgcttggtggaaccctgctgccggggtcactcctgaggagcgtgactggctggaagaaaagtatccaggatggaataaacgttggggtcgttgctgggatgtgatcaccgaaaacgttctcaatgaccgtatggatcttgtctctccagaaaccttgcccagcgtgtgcaacatgagccagataccgctggtaggtgttcctggtgatgactggaatatcgaagttttcagtcttgagcacaatgggcgtctttatcattttggctctgaagtggatcgctgggtattccagcaagatccggttcagtatcaaaatcatatgaatatcgtcgaccgcttcctcgcaggtcagatacagccgatgactttggaaggtgccctcaaatatatgggcttccaatctattgaagagatgggcaaagacgcccacgactttgcatgggctgacaagtgcaagcctgctatgaagaaatcggcctga

SEQ ID NO: 25 - protein, 500 aa, Pseudomonas mendocina KR1
MAMHPRKDWYELTRATNWTPSYVTEEQLFPERMSGHMGIPLEKWESYDEPYKTSYPEYVSIQREKDAGAYSVKAALERAKIYENSDPGWISTLKSHYGAIAVGEYAAVTGEGRMARFSKAPGNRNMATFGMMDELRHGQLQLFFPHEYCKKDRQFDWAWRAYHSNEWAAIAAKHFFDDIITGRDAISVAIMLTFSFETGFTNMQFLGLAADAAEAGDYTFANLISSIQTDESRHAQQGGPALQLLIENGKREEAQKKVDMAIWRAWRLFAVLTGPVMDYYTPLEDRSQSFKEFMYEWIIGQFERSLIDLGLDKPWYWDLFLKDIDELHHSYHMGVWYWRTTAWWNPAAGVTPEERDWLEEKYPGWNKRWGRCWDVITENVLNDRMDLVSPETLPSVCNMSQIPLVGVPGDDWNIEVFSLEHNGRLYHFGSEVDRWVFQQDPVQYQNHMNIVDRFLAGQIQPMTLEGALKYMGFQSIEEMGKDAHDFAWADKCKPAMKKSA

SEQ ID NO: 26 - DNA, 984 bp, Pseudomonas mendocina KR1
atgagctttgaatccaagaaaccgatgcgtacatggagccacctggccgaaatgagaaagaagccaagtgagtacgatattgtctcacgcaagcttcactacagtaccaacaatcccgattcaccctgggagctgagccccgatagcccaatgaatctgtggtacaagcagtaccgtaacgcatcgccattgaaacacgataactgggatgcttttactgatcctgaccaacttgtataccgcacctacaacctgatgcaggatggtcaggaatcttatgtgcagagtctgttcgatcaattcaatgagcgcgaacatgaccaaatggtgcgggagggctgggagcacacaatggcccgctgttattccccgttgcgctatctgttccactgcctgcagatgtcgtcggcctatgttcagcagatggcgccggcgagcacaatctcaaattgctgcatccttcaaactgctgacagcctgcgatggttgacgcacaccgcctaccgaacgcacgaactcagtcttacttatccggatgctggtttaggtgagcacgagcgagaactgtgggagaaagagccgggttggcaggggctgcgtgaattgatggagaagcaactaactgcttttgattggggagaggcttttgtcagtctaaatttggtggtcaagccaatgattgtcgagagtattttcaaaccactgcagcagcaagcatgggaaaataacgataccttgcttcctctgttgattgacagtcagctgaaagatgccgagcgtcatagtcgttggtcgaaagcacttgtaaaacatgcgctggaaaaccccgataatcacgctgtaattgaaggttggattgaaaagtggcgccccttggctgacagggcagctgaagcttacctgagtatgctatctagcgacattttgcccgctcaatatcttgagcgtagtacctcattgagggcatccatacttacggtctga

SEQ ID NO: 27 - protein, 327 aa, Pseudomonas mendocina KR1
MSFESKKPMRTWSHLAEMRKKPSEYDIVSRKLHYSTNNPDSPWELSPDSPMNLWYKQYRNASPLKHDNWDAFTDPDQLVYRTYNLMQDGQESYVQSLFDQFNEREHDQMVREGWEHTMARCYSPLRYLFHCLQMSSAYVQQMAPASTISNCCILQTADSLRWLTHTAYRTHELSLTYPDAGLGEHERELWEKEPGWQGLRELMEKQLTAFDWGEAFVSLNLVVKPMIVESIFKPLQQQAWENNDTLLPLLIDSQLKDAERHSRWSKALVKHALENPDNHAVIEGWIEKWRPLADRAAEAYLSMLSSDILPAQYLERSTSLRASILTV

SEQ ID NO: 28 - DNA, 255 bp, Pseudomonas mendocina KR1
atgtcggcatttccagttcacgcagcgtttgaaaaagatttcttggttcaactggtagtggtggatttaaatgattccatggaccaggtagcggagaaagttgcctaccattgtgttaatcgtcgtgttgctcctcgtgaaggtgtcatgcgggttcgaaagcatagatcaactgagctatttccacgggatatgaccatagctgagagcggccttaacccaactgaagtgatcgatgtggtattcgaggagtag

SEQ ID NO: 29 - protein, 84 aa, Pseudomonas mendocina KR1
MSAFPVHAAFEKDFLVQLVVVDLNDSMDQVAEKVAYHCVNRRVAPREGVMRVRKHRSTELFPRDMTIAESGLNPTEVIDVVFEE

SEQ ID NO: 30 - DNA, 789 bp, Pseudomonas aeruginosa
atgaaaacgacgcagtacgtggcccgccagcccgacgacaacggtttcatccactatccggaaaccgagcaccaggtctggaataccctgatcacccggcaactgaaggtgatcgaaggccgcgcctgtcaggaatacctcgacggcatcgaacagctcggcctgccccacgagcggatcccccagctcgacgagatcaacagggttctccaggccaccaccggctggcgcgtggcacgggttccggcgctgattccgttccagaccttcttcgaactgctggccagccagcaattccccgtcgccaccttcatccgcaccccggaagaactggactacctgcaggagccggacatcttccacgagatcttcggccactgcccactgctgaccaacccctggctcgccgagttcacccatacctacggcaagctcggcctcaaggcgagcaaggaggaacgcgtgttcctcgcccgcctgtactggatgaccatcgagttcggcctggtcgagaccgaccagggcaagcgcatctacggcggcggcatcctctcctcgccgaaggagaccgtctacagcctctccgacgagccgctgcaccaggccttcaatccgctggaggcgatgcgcacgccctaccgcatcgacatcctgcaaccgctctatttcgtcctgcccgacctcaagcgcctgttccaactggcccaggaagacatcatggcgctggtccacgaggccatgcgcctgggcctgcacgcgccgctgttcccgcccaagcaggcggcctga

SEQ ID NO: 31 - protein, 262 aa, Pseudomonas aeruginosa
MKTTQYVARQPDDNGFIHYPETEHQVWNTLITRQLKVIEGRACQEYLDGIEQLGLPHERIPQLDEINRVLQATTGWRVARVPALIPFQTFFELLASQQFPVATFIRTPEELDYLQEPDIFHEIFGHCPLLTNPWLAEFTHTYGKLGLKASKEERVFLARLYWMTIEFGLVETDQGKRIYGGGILSSPKETVYSLSDEPLHQAFNPLEAMRTPYRIDILQPLYFVLPDLKRLFQLAQEDIMALVHEAMRLGLHAPLFPPKQAA

SEQ ID NO: 32 - DNA, 357 bp, Pseudomonas aeruginosa
atgaccgcactcacccaagcccattgcgaagcctgccgcgcagacgccccgcacgtcagcgacgaagaactgcccgtgctgctgcggcaaatcccggattggaacatcgaagtccgcgacggcatcatgcagctagagaaggtctacctgttcaagaacttcaagcatgccctggccttcaccaatgccgtcggcgagatatccgaggccgaaggccaccatccgggcctgctgaccgagtggggcaaagtcaccgtgacctggtggagccactcgatcaagggcctgcaccgcaacgatttcatcatggcggcgcgcaccgatgaggtagcgaaaaccgccgaggggcgcaaatga

SEQ ID NO: 33 - protein, 118 aa, Pseudomonas aeruginosa
MTALTQAHCEACRADAPHVSDEELPVLLRQIPDWNIEVRDGIMQLEKVYLFKNFKHALAFTNAVGEISEAEGHHPGLLTEWGKVTVTWWSHSIKGLHRNDFIMAARTDEVAKTAEGRK

SEQ ID NO: 34 - DNA, 789 bp, Pseudomonas putida
atgaaacagacgcaatacgtggcacgcgagcccgatgcgcatggttttatcgattacccgcagcaagagcatgcggtgtggaacaccctgatcacccgccagctgaaagtgatcgaaggccgtgcgtgccaggaatacctggacggcatcgaccagctgaaattgccgcatgaccgcattccgcaactgggcgagatcaacaaggtgctgggtgccaccaccggctggcaggttgcccgggttccggcgctgatccccttccagaccttcttcgaattgctggccagcaagcgctttccggtcgccaccttcatccgcaccccggaagagctggactacctgcaagagccggatatcttccacgagatcttcggccactgcccgctgctgaccaatccctggttcgccgaattcacccacacctacggcaagctcggcctggccgcgaccaaggaacaacgtgtgtacctggcacgcttgtactggatgaccatcgagtttggcctgatggaaaccgcgcaaggccgcaaaatctatggtggtggcatcctctcgtcgccgaaagagaccgtctacagtctgtctgacgagcctgagcaccaggccttcgacccgatcgaggccatgcgtacaccctaccgcatcgacattctgcaaccggtgtatttcgtactgccgaacatgaagcgcctgttcgacctggcccacgaggacatcatgggcatggtccataaagccatgcagctgggtctgcatgcaccgaagtttccacccaaggtcgctgcctga

SEQ ID NO: 35 - protein, 262 aa, Pseudomonas putida
MKQTQYVAREPDAHGFIDYPQQEHAVWNTLITRQLKVIEGRACQEYLDGIDQLKLPHDRIPQLGEINKVLGATTGWQVARVPALIPFQTFFELLASKRFPVATFIRTPEELDYLQEPDIFHEIFGHCPLLTNPWFAEFTHTYGKLGLAATKEQRVYLARLYWMTIEFGLMETAQGRKIYGGGILSSPKETVYSLSDEPEHQAFDPIEAMRTPYRIDILQPVYFVLPNMKRLFDLAHEDIMGMVHKAMQLGLHAPKFPPKVAA

SEQ ID NO: 36 - DNA, 357 bp, Pseudomonas putida
atgaatgccttgaaccaagcccattgcgaagcctgccgcgccgacgcaccgaaagtctccgacgaagagctggccgagctgattcgcgaaatcccggactggaacattgaagtacgtgacggccacatggagcttgagcgcgtgttcctgttcaagaacttcaagcacgccttggcgttcaccaacgccgtgggcgaaatcgccgaagccgaaggccaccacccagggctgctgaccgagtggggcaaggttaccgtcacttggtggagccactcgatcaaaggcctgcaccgcaacgacttcatcatgtgcgcgcgcactgacaaggtggctgaatcggctgaaggccgtaagtaa

SEQ ID NO: 37 - protein, 118 aa, Pseudomonas putida
MNALNQAHCEACRADAPKVSDEELAELIREIPDWNIEVRDGHMELERVFLFKNFKHALAFTNAVGEIAEAEGHHPGLLTEWGKVTVTWWSHSIKGLHRNDFIMCARTDKVAESAEGRK

SEQ ID NO: 38 - DNA, 1707 bp, Agaricus bisporus
atgtctcatctgctcgtttctcctcttggaggaggcgttcaacctcgtcttgaaataaataattttgtaaagaatgaccgtcaattctctctttacgttcaagctctcgaccggatgtacgccacccctcagaatgaaactgcgtcctactttcaagtagctggagtgcatggatacccactcatccctttcgatgatgcagtcggtccaaccgagttcagtccttttgaccaatggactgggtattgcactcacggctcaactctttttccaacttggcatcgtccttatgttttgattctcgaacaaattttgagtggacacgctcaacaaatcgccgatacttacactgtcaataaatccgagtggaaaaaggcggcaaccgaattccgtcatccgtattgggattgggcatctaatagcgttcctcctccggaagtcatctccctacccaaagtcactatcacgactccgaatggccaaaagacgagcgtcgccaacccactgatgaggtatactttcaactctgtcaacgacggcggtttctatgggccgtataatcagtgggatactactttgagacaacccgactcgacgggtgtgaacgcaaaggataacgttaataggcttaaaagtgttttgaaaaatgctcaagccagtcttacacgggctacttacgacatgttcaaccgcgtcacgacttggcctcatttcagcagccatactcctgcgtctggaggaagtaccagtaatagtatcgaggcaattcatgacaatatccatgtcctcgtcggtggtaacggccacatgagtgatccttctgtcgccccctttgatcctatcttcttcttgcatcatgcgaacgttgatcgactgattgctttatggtcggctattcgttacgatgtgtggacttccccgggcgacgctcaatttggtacatatactttgagatataagcagagtgttgacgagtcgaccgaccttgctccgtggtggaagactcaaaatgaatactggaaatccaatgaactgaggagcaccgagtcgttgggatacacttaccccgagtttgttggtttggatatgtacaacaaagacgcggtaaacaagaccatttcccgaaaggtagcacagctttatggaccacaaagaggagggcaaaggtcgctcgtagaggatttatcaaactcccatgctcgtcgtagtcaacgccctgcgaagcgctcccgccttggtcaactcttgaaagggttattctcggattggtctgctcaaatcaaattcaaccgccatgaagtcggccagagcttctcggtttgtcttttcctgggcaatgttcctgaagacccgagggagtggttggttagccccaacttggttggcgctcgtcatgcgttcgtccgttcggtcaagaccgaccatgtagccgaggaaataggtttcattccgattaaccagtggattgccgagcacacgggtttaccttcgtttgcagtagaccttgtaaaaccactcttggcacaaggtttacagtggcgcgtgctcttggcggatggaacccctgctgagctcgattcactggaagtgactatattggaggtcccatccgagctgaccgacgatgagcctaatccccgctccaggccgcccaggtaccacaaggatattacacacggaaagcgtggtggttgccgcgaggcttga

SEQ ID NO: 39 - protein, 568 aa, Agaricus bisporus
MSHLLVSPLGGGVQPRLEINNFVKNDRQFSLYVQALDRMYATPQNETASYFQVAGVHGYPLIPFDDAVGPTEFSPFDQWTGYCTHGSTLFPTWHRPYVLILEQILSGHAQQIADTYTVNKSEWKKAATEFRHPYWDWASNSVPPPEVISLPKVTITTPNGQKTSVANPLMRYTFNSVNDGGFYGPYNQWDTTLRQPDSTGVNAKDNVNRLKSVLKNAQASLTRATYDMFNRVTTWPHFSSHTPASGGSTSNSIEAIHDNIHVLVGGNGHMSDPSVAPFDPIFFLHHANVDRLIALWSAIRYDVWTSPGDAQFGTYTLRYKQSVDESTDLAPWWKTQNEYWKSNELRSTESLGYTYPEFVGLDMYNKDAVNKTISRKVAQLYGPQRGGQRSLVEDLSNSHARRSQRPAKRSRLGQLLKGLFSDWSAQIKFNRHEVGQSFSVCLFLGNVPEDPREWLVSPNLVGARHAFVRSVKTDHVAEEIGFIPINQWIAEHTGLPSFAVDLVKPLLAQGLQWRVLLADGTPAELDSLEVTILEVPSELTDDEPNPRSRPPRYHKDITHGKRGGCREA

SEQ ID NO: 40 - DNA, 1671 bp, Agaricus bisporus
atgtcgctgattgctactgtcggacctactggcggagtcaagaaccgtctgaacatcgttgattttgtgaagaatgaaaagtttttcacgctttatgtacgctccctcgaacttctacaagccaaggaacagcatgactactcgtctttcttccaactagccggcattcatggtctaccctttactgagtgggccaaagagcgaccttccatgaacctatacaaggctggttattgtacccatgggcaggttctgttcccgacttggcatagaacgtacctttctgtgttggagcaaatacttcaaggagctgccatcgaagttgctaagaagttcacttctaatcaaaccgattgggtccaggcggcgcaggatttacgccagccctactgggattggggtttcgaacttatgcctcctgatgaggttatcaagaacgaagaggtcaacattacgaactacgatggaaagaagatttccgtcaagaaccctatcctccgctatcacttccatccgatcgatccttctttcaagccatacggggactttgcaacctggcgaacaacagtccgaaaccccgatcgtaataggcgagaggatatccctggtctaatcaaaaaaatgagacttgaggaaggtcagattcgtgagaagacctacaatatgttgaagttcaacgatgcttgggagagattcagtaaccacggcatatctgatgatcagcatgctaacagcttggagtctgttcacgatgacattcatgttatggttggatacggcaaaatcgaaggacatatggaccaccctttctttgctgccttcgacccgattttctggttacatcataccaacgtcgaccgtctactatccctttggaaagcaatcaaccccgatgtgtgggttacgtcgggacgtaaccgggatggtaccatgggcatcgcacccaacgctcagatcaacagcgagacccctcttgagccattctaccaatctggggataaagtgtggacctcggcctctctcgctgatactgctcggctcggctactcctaccccgatttcgacaagttggttggaggaacaaaggagttgattcgcgacgctatcgacgacctcatcgatgagcggtatggaagcaaaccttcgagtggggctcgcaatactgcctttgatctcctcgccgatttcaagggcattaccaaagagcacaaggaggatctcaaaatgtacgactggaccatccatgttgccttcaagaagttcgagttgaaagagagtttcagtcttctcttctactttgcgagtgatggtggcgattatgatcaggagaattgctttgttggatcaattaacgccttccgtgggactgctcccgaaacttgcgcgaactgccaagataacgagaacttgattcaagaaggctttattcacttgaatcattatcttgctcgtgaccttgaatctttcgagccgcaggacgtgcacaagttcttaaaggaaaaaggactgtcatacaaactctacagcaggggagataaacctttgacatcgttgtcagttaagattgaaggacgtccccttcatctaccgcccggagagcatcgtccgaagtacgatcacactcaggcccgagtagtgtttgatgatgtcgcggtgcatgttattaactga

SEQ ID NO: 41 - protein, 556 aa, Agaricus bisporus
MSLIATVGPTGGVKNRLNIVDFVKNEKFFTLYVRSLELLQAKEQHDYSSFFQLAGIHGLPFTEWAKERPSMNLYKAGYCTHGQVLFPTWHRTYLSVLEQILQGAAIEVAKKFTSNQTDWVQAAQDLRQPYWDWGFELMPPDEVIKNEEVNITNYDGKKISVKNPILRYHFHPIDPSFKPYGDFATWRTTVRNPDRNRREDIPGLIKKMRLEEGQIREKTYNMLKFNDAWERFSNHGISDDQHANSLESVHDDIHVMVGYGKIEGHMDHPFFAAFDPIFWLHHTNVDRLLSLWKAINPDVWVTSGRNRDGTMGIAPNAQINSETPLEPFYQSGDKVWTSASLADTARLGYSYPDFDKLVGGTKELIRDAIDDLIDERYGSKPSSGARNTAFDLLADFKGITKEHKEDLKMYDWTIHVAFKKFELKESFSLLFYFASDGGDYDQENCFVGSINAFRGTAPETCANCQDNENLIQEGFIHLNHYLARDLESFEPQDVHKFLKEKGLSYKLYSRGDKPLTSLSVKIEGRPLHLPPGEHRPKYDHTQARVVFDDVAVHVIN

SEQ ID NO: 42 - DNA, 1122 bp, Escherichia coli
atggttgctgaattgaccgcattacgcgatcaaattgatgaagtcgataaagcgctgctgaatttattagcgaagcgtctggaactggttgctgaagtgggcgaggtgaaaagccgctttggactgcctatttatgttccggagcgcgaggcatctatgttggcctcgcgtcgtgcagaggcggaagctctgggtgtaccgccagatctgattgaggatgttttgcgtcgggtgatgcgtgaatcttactccagtgaaaacgacaaaggatttaaaacactttgtccgtcactgcgtccggtggttatcgtcggcggtggcggtcagatgggacgcctgttcgagaagatgctgaccctctcgggttatcaggtgcggattctggagcaacatgactgggatcgagcggctgatattgttgccgatgccggaatggtgattgttagtgtgccaatccacgttactgagcaagttattggcaaattaccgcctttaccgaaagattgtattctggtcgatctggcatcagtgaaaaatgggccattacaggccatgctggtggcgcatgatggtccggtgctggggctacacccgatgttcggtccggacagcggtagcctggcaaagcaagttgtggtctggtgtgatggacgtaaaccggaagcataccaatggtttctggagcaaattcaggtctggggcgctcggctgcatcgtattagcgccgtcgagcacgatcagaatatggcgtttattcaggcactgcgccactttgctacttttgcttacgggctgcacctggcagaagaaaatgttcagcttgagcaacttctggcgctctcttcgccgatttaccgccttgagctggcgatggtcgggcgactgtttgctcaggatccgcagctttatgccgacatcattatgtcgtcagagcgtaatctggcgttaatcaaacgttactataagcgtttcggcgaggcgattgagttgctggagcagggcgataagcaggcgtttattgacagtttccgcaaggtggagcactggttcggcgattacgcacagcgttttcagagtgaaagccgcgtgttattgcgtcaggcgaatgacaatcgccagtaa

SEQ ID NO: 43 - protein, 373 aa, Escherichia coli
MVAELTALRDQIDEVDKALLNLLAKRLELVAEVGEVKSRFGLPIYVPEREASMLASRRAEAEALGVPPDLIEDVLRRVMRESYSSENDKGFKTLCPSLRPVVIVGGGGQMGRLFEKMLTLSGYQVRILEQHDWDRAADIVADAGMVIVSVPIHVTEQVIGKLPPLPKDCILVDLASVKNGPLQAMLVAHDGPVLGLHPMFGPDSGSLAKQVVVWCDGRKPEAYQWFLEQIQVWGARLHRISAVEHDQNMAFIQALRHFATFAYGLHLAEENVQLEQLLALSSPIYRLELAMVGRLFAQDPQLYADIIMSSERNLALIKRYYKRFGEAIELLEQGDKQAFIDSFRKVEHWFGDYAQRFQSESRVLLRQANDNRQ

(19) United States
(12) Patent Application Publication
(10) Pub. No.: US 2010/0143990 A1
(43) Pub. Date: Jun. 10, 2010

(54) FERMENTATIVE PRODUCTION OF HYDROXYTYROSOL

(76) Inventors: Jühane Achkar, Zürich (CH); Abel Ferrandez, Basel (CH)

Correspondence Address: NIXON & VANDERHYE, PC, 901 NORTH GLEBE ROAD, 11TH FLOOR, ARLINGTON, VA, 22203, US

(21) Appl. No.: 12/516,143

(22) PCT Filed: Nov. 27, 2007
(86) PCT No.: PCT/EP2007/010208

§ 371 Date: Sep. 1, 2009

(30) Foreign Application Priority Data
Nov. 27, 2006 (EP) 06024493.6

(51) Int. Cl. C12P 7/22; C12N 1/00

(52) U.S. Cl. 435/156; 435/243

(57) ABSTRACT

The present invention relates to a newly identified microorganisms capable of direct production of hydroxytyrosol (hereinafter also referred to as Hy-T) from a carbon source obtainable from the D-glucose metabolization pathway. The invention also relates to polynucleotide sequences comprising genes that encode proteins which are involved in the synthesis of Hy-T. The invention also relates to genetically engineered microorganisms and their use for the direct production of Hy-T.

The present invention relates to genetically altered microorganisms and their use for the direct production of hydroxytyrosol. The invention also relates to the use of polynucleotides and polypeptides as biotechnological tools in the production of hydroxytyrosol from microorganisms, whereby said polynucleotides and/or encoded polypeptides have a direct or indirect impact on yield, production, and/or efficiency of production of the fermentation product.

Hydroxytyrosol (hereafter called Hy-T) is a potent antioxidant found in olives, thus present in high abundance in olive mill waste waters. Hy-T has been associated with the lower mortality and incidence of cancer in Mediterranean regions and has been attributed cardio protective properties. There has been therefore an increased interest in the manufacturing and commercialization of Hy-T as nutritional supplement.

Currently, hydroxytyrosol is commercially available only in the form of enriched olive extracts.

Methods for the chemical synthesis of Hy-T have been described, but they make use of environmentally hazardous products such as organic solvents, strong acids, hydrides and/or cyanides. Therefore, over the past years, other approaches to manufacture Hy-T using different extraction methods and/or microbial conversions, which would be more economical as well as ecological, have been investigated.

For example, EP-A-1,623,960 teaches on the recovery of a structural analogue of Hy-T such as tyrosol from olive mill wastewaters via expensive procedures such as microfiltration, ultrafiltration, nanofiltration and reverse osmosis followed by oxidation with heavy metal based catalysts. Further Bouzid O., et al. (Proc. Biochem. (2005) 40: 1855-1862) discloses a method to enrich oil by-products in Hy-T by their treatment with cells of Aspergillus niger enriched in cinnamoyl esterases. Several other examples for the extraction of Hy-T from olive oil, olive tree leaves or olive oil production waste waters can be found, these procedures being developed at low yields, requiring expensive extraction processes and the use of toxic compounds such as organic solvents, or hazardous strong acid treatments.

Further, WO/02/16628 discloses a method for the transformation of tyrosol in vitro making use of purified mushroom tyrosinase. This enzymatic procedure has as main disadvantages the elevated cost of a purified enzyme, as well as the intrinsic instability of enzymes isolated from their natural cellular environment. Furthermore, reaction conditions in this method are restricted to phosphate solutions buffered at pH 7, and the use of room temperature, making use of costly protein removing systems such as molecular size discriminating membranes and purification methods based on techniques such as high performance liquid chromatography (HPLC) of high cost for industrial application purposes. It is therefore desirable to make use of technologies offering a broader range of reaction conditions for their applicability and not restricting themselves to the use of purified mushroom tyrosinase. No enzyme other than mushroom tyrosinase is found in the prior art capable of transforming organic compounds such as, for example, tyrosol to Hy-T.

Finally, the ability to transform the precursor tyrosol to hydroxytyrosol has been reported in a few microorganisms, but there is no previous report indicating how to increase the ability of microorganisms to transform organic compounds such as, for example, tyrosol to Hy-T. Furthermore, one of the main disadvantages of the approaches cited above is the use of undesirable human opportunistic pathogens such as Pseudomonas aeruginosa (Allouche N., et al. Appl. Environ. Microbiol. (2004) 70: 2105-2109) or Serratia marcensces (Allouche N., et al. J. Agric. Food Chem. (2005) 53: 6525-6530). Furthermore, these organisms are described as not only capable of transforming tyrosol to Hy-T, but also of utilizing the costly and highly valuable substrate tyrosol as carbon source i.e. of eliminating the substrate and its product Hy-T from the culture medium. Although prior art teaches how to transform tyrosol (2-(4-hydroxyphenyl)ethanol) to Hy-T, surprisingly there is no known biotechnological method described so far for the transformation of organic compounds other than tyrosol to Hy-T.

Consequently, there is a need to develop optimized fermentation systems for the microbial production of Hy-T making use of renewable resources.

It has now been found that two groups of enzymes involved in the catabolism of aromatic compounds play an important role in the biotechnological production of Hy-T. It has also been found, that by using polynucleotide sequences encoding these enzymes in a microorganism, such as for example Escherichia coli, the fermentation for Hy-T from a carbon source obtainable from the pathway of D-glucose metabolism of said microorganism can be even greatly improved.

More precisely, it has been found that the enzymes capable to improve fermentative production of Hy-T are involved either in the design of the Hy-T specific hydroxylation pattern (HP enzymes) of aromatic compounds or of the correct functional group of Hy-T (FG enzymes). Polynucleotides according to the invention and proteins encoded by these polynucleotides are herein abbreviated by HP and FG.

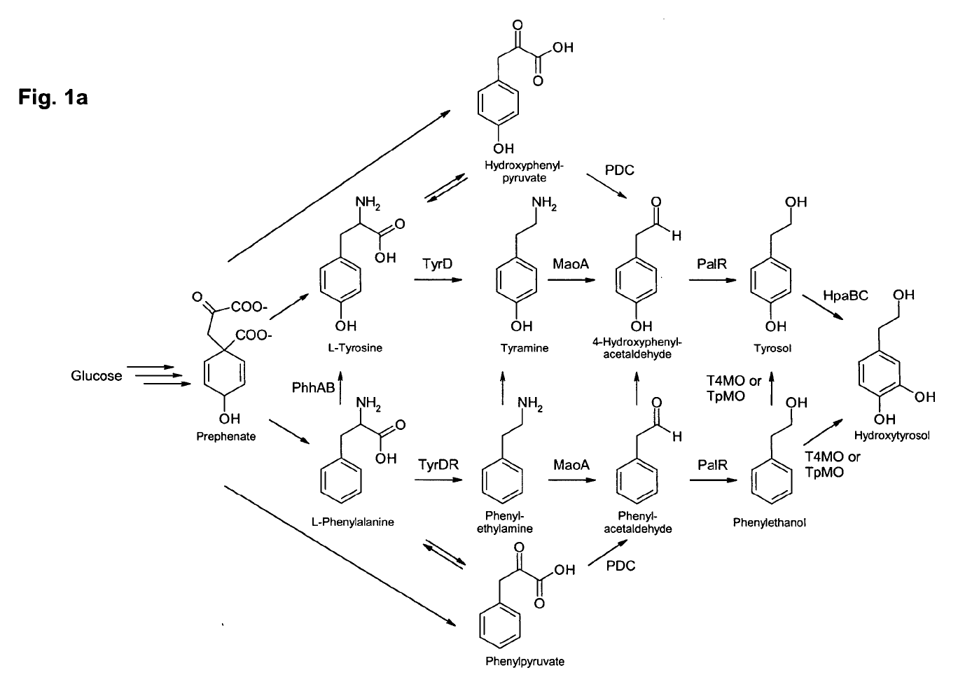

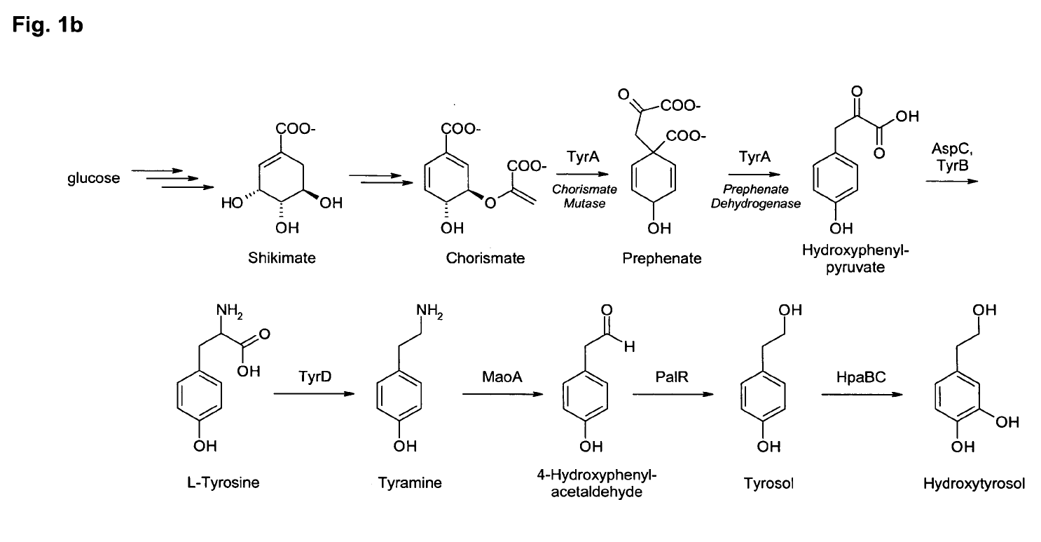

The enzymes involved in the biosynthesis of hydroxytyrosol and which are capable of improving Hy-T production are shown in FIGS. 1a and 1b.

HP and FG encoding polynucleotides are known in the art. The candidates which are able to improve fermentative production of Hy-T according to the present invention are selected from the group consisting of:
1. Polynucleotides encoding enzymes capable of transforming tyrosol into Hy-T and/or L-tyrosine into L-3,4-dihydroxyphenylalanine comprising the polynucleotide sequence according to SEQ ID NO:1; SEQ ID NO:38 and SEQ ID NO:40 or variants thereof. SEQ ID NO:1 corresponds to a tyrosinase from Pycnoporus sanguineus, a HP enzyme according to SEQ ID NO:2. SEQ ID NO:38 and SEQ ID NO 40 correspond to two tyrosinases from Agaricus bisporus, HP enzymes according to SEQ ID NO:39 and SEQ ID NO: 41.
2. Polynucleotides encoding enzymes capable of transforming phenylacetaldehyde to phenylethanol and/or 4-hydroxyphenylacetaldehyde to tyrosol comprising the polynucleotide sequence according to SEQ ID NO:3 or variants thereof.
   SEQ ID NO:3 corresponds to the gene palR gene from Rhodococcus elythropolis which encodes a phenylacetaldehyde reductase (PalR), a FG-enzyme according to SEQ ID NO:4, that catalyzes the asymmetric reduction of aldehydes or ketones to chiral alcohols. This NADH-dependent enzyme belongs to the family of zinc-containing medium-chain alcohol dehydrogenases.
3. Polynucleotides encoding enzymes capable of transforming tyrosol to Hy-T comprising the polynucleotide sequence according to SEQ ID NO:5 and/or SEQ ID NO:7 or variants thereof.
   The hpaB and hpaC genes from Escherichia coli W which correspond to SEQ ID NO:5 and SEQ ID NO:7 respectively express a two-components enzyme, 4-hydroxyphenylacetate 3-monooxygenase. The HP-enzyme (HpaBC) was reported to be a two-component flavin-dependent monooxygenase that catalyzes the hydroxylation of 4-hydroxyphenylacetate into 3,4-dihydroxyphenylacetate. The large component (HpaB; protein SEQ ID NO:6,) is a reduced flavin-utilizing monooxygenase. The small component (HpaC, protein SEQ ID NO:8) is an oxido-reductase that catalyzes flavin reduction using NAD(P)H as a reducent.
4. Polynucleotides encoding enzymes capable of transforming L-phenylalanine to 2-phenylethylamine and/or L-tyrosine to tyramine comprising the polynucleotide sequence according to SEQ ID NO:9 or variants thereof.
   SEQ ID NO:9 corresponds to the gene tyrDR from Pseudomonas putida which encodes an FG-enzyme belonging to the enzymatic family of aromatic-L-amino-acid decarboxylases, such as, for example, L-phenylalanine and L-tyrosine decarboxylases according to SEQ ID NO:10.
5. Polynucleotides encoding enzymes capable of transforming 2-phenylethylamine to phenylacetaldehyde and/or tyramine to 4-hydroxyphenylacetaldehyde comprising the polynucleotide sequence according to SEQ ID NO:11 or variants thereof
   SEQ ID NO:11 corresponds to the maoA gene from E. coli K-12 which encodes a monoamine oxidase (MaoA), a copper-containing FG-enzyme according to SEQ ID NO:12 using 3,4,6-trihydroxyphenylalanine quinone as cofactor that catalyzes the oxidative deamination of monoamines to produce the corresponding aldehyde. Oxygen is used as co-substrate with the amine, and ammonia and hydrogen peroxide are by-products of the reaction in addition to the aldehyde.
6. Polynucleotides encoding enzymes capable of transforming L-tyrosine to tyramine comprising the polynucleotide sequence according to SEQ ID NO:13 or variants thereof
   SEQ ID NO:13 corresponds to the tyrD gene which encodes a tyrosine decarboxylase (TyrD) from Methanocaldococcus jannaschii according to SEQ ID NO:14, a lyase which is an FG-enzyme that catalyzes the removal of the carboxylate group from the amino acid tyrosine to produce the corresponding amine tyramine and carbon dioxide using pyridoxal 5′-phosphate as a necessary cofactor.
7. Polynucleotides encoding enzymes capable of transforming phenylpyruvate to phenylacetaldehyde and/or hydroxyphenylpyruvate to 4-hydroxyphenylactealdehyde comprising the polynucleotide sequence according to SEQ ID NO:16 or variants thereof SEQ ID NO:16 corresponds to the PDC gene from Acinetobacter calcoaceticus which encodes an FG-enzyme (SEQ ID NO:17) that has the activity of a phenylpyruvate decarboxylase.
8. Polynucleotides encoding hydroxylating enzymes such as toluene monooxygenases which are capable of transforming phenylethanol to Hy-T and/or tyrosol. For example, toluene para-monooxygenase (TpMO) from Ralstonia pickettii PK01 and toluene 4-monooxygenase (T4MO) from Pseudomonas mendocina KR1. Both enzymes are multi-component non-heme diiron monooxygenases encoded by six genes and comprising a hydroxylase component structured in three alpha-, beta-, and gamma-subunits that assemble into an HP-enzyme.
   SEQ ID NO:18, 20 and 22 encode the alpha, beta and gamma subunits of TpMO, respectively, and SEQ ID NO: 19, 21 and 23 represent the protein sequences of these subunits, respectively.
   SEQ ID NO:24, 26 and 28 encode the alpha, beta and gamma subunits of T4MO, respectively, and SEQ ID NO 25, 27 and 29 represent the protein sequences of these subunits, respectively.
9. Polynucleotides encoding enzymes capable of transforming L-phenylalanine to L-tyrosine comprising the polynucleotide sequences according to
   SEQ ID NO:30 and/or SEQ ID NO:32; or
   SEQ ID NO:34 and/or SEQ ID NO:36
   or variants thereof.
   These two pairs of sequences correspond to the phhAB genes which encodes a two-component hydroxylase (HP-enzyme). The large component (PhhA) represented by SEQ ID NO:30 and SEQ ID NO:34 encode the proteins according to SEQ ID NO:31 and SEQ ID NO:35, respectively, which are phenylalanine-4-hydroxylase enzymes from P. aeruginosa and P. putida, respectively. The small component (PhhB) represented by SEQ ID NO:32 and SEQ ID NO:36 encode the proteins according to SEQ ID NO:33 and SEQ ID NO 37, respectively, which are pterin-4-alpha-carbinolamine dehydratase enzymes from P. aeruginosa and P. putida, respectively.
10. Polynucleotides encoding enzymes involved in the transformation of chorismate to prephenate and/or prephenate into hydroxyphenylpyruvate comprising the polynucleotide sequence according to SEQ ID NO:42 or variants thereof. SEQ ID NO:42 corresponds to the tyrA gene from E. coli K-12 which encodes an FG-enzyme (SEQ ID NO:43) that has the activity of a chorismate mutase and prephenate dehydrogenase.

It is now the object of the present invention to provide a process for the direct fermentative production of Hy-T from glucose. by using a genetically engineered host cell which expresses polynucleotides encoding an enzyme capable of transforming tyrosol to Hy-T and at least one polynucleotide encoding an enzyme which has an activity selected from the group consisting of:
phenylacetaldehyde reductase activity,
L-phenylalanine and/or L-tyrosine decarboxylase activity,
monoamine oxidase activity,
a lyase activity,
phenylpyruvate decarboxylase activity,
toluene monooxygenase, for example, toluene para-monooxygenase activity,
phenylalanine-4-hydroxylase and/or pterin-4-alpha-carbinolamine dehydratase activity,
chorismate mutase and/or prephenate dehydrogenase activity.

Furthermore, it is also an object of the present invention to provide a process for producing a host cell which is genetically engineered, for example transformed by such polynucleotide (DNA) sequences or vectors comprising polynucleotides as defined above. This may be accomplished, for example, by transferring polynucleotides as exemplified herein into a recombinant or non-recombinant host cell that may or may not contain an endogenous equivalent of the corresponding gene.

Such a transformed cell is also an object of the invention.

Advantageous embodiments of the invention become evident from the dependent claims. These and other aspects and embodiments of the present invention should be apparent to those skilled in the art from the teachings herein.

The term “direct fermentation”, “direct production”, “direct conversion”, “direct bioconversion”, “direct biotransformation” and the like is intended to mean that a microorganism is capable of the conversion of a certain substrate into the specified product by means of one or more biological conversion steps, without the need of any additional chemical conversion step. A single microorganism capable of directly fermenting Hy-T is preferred.

As used herein, “improved” or “improved yield of Hy-T” or “higher yield” or “improved bioconversion ratio” or “higher bioconversion ratio” caused by a genetic alteration means an increase of at least 5%, 10%, 25%, 30%, 40%, 50%, 75%, 100%, 200% or even more than 500%, compared to a cell which is not genetically altered. Such unaltered cells are also often referred to as wild type cells.

The term “genetically altered” or “genetically engineered” means any mean of changing the genetic material of a living organism. It can involve the production and use of recombinant DNA, but other methods are available and are known to those skilled in the art to produce genetically altered microorganisms such as, for example, but not limited to, chemical treatments or exposure to ultraviolet or X-Ray irradiation. More in particular it is used to delineate the genetically engineered or modified organism from the naturally occurring organism. Genetic engineering may be done by a number of techniques known in the art, such as e.g. gene replacement, gene amplification, gene disruption, transfection, transformation using plasmids, viruses, or other vectors. A genetically modified organism, e.g. genetically modified microorganism, is also often referred to as a recombinant organism, e.g. recombinant microorganism.

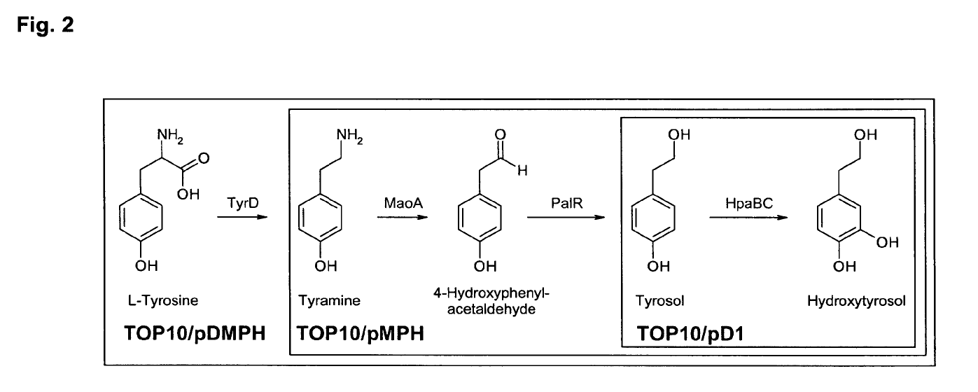

In a preferred embodiment of the invention at least three or four or five or six polynucleotides encoding a protein selected from the groups defined above, are transferred into a recombinant or non-recombinant microorganism—hereinafter also called host cell—in such a way that the host cell is able to produce Hy-T directly from glucose as carbon source. Preferred polynucleotides for such combinations are hpaBC, maoA, palR, tyrD, TyrDR and TyrA. The enzyme reactions carried out by the corresponding polypeptides HpaBC, MaoA, PalR, TyrDand TyrA are described in FIG. 2.

Any cell that serves as recipient of the foreign nucleotide acid molecules may be used as a host cell, such as for instance a cell carrying a replicable expression vector or cloning vector or a cell being genetically engineered or genetically altered by well known techniques to contain desired gene(s) on its chromosome(s) or genome. The host cell may be of prokaryotic or eukaryotic origin, such as, for instance bacterial cells, animal cells, including human cells, fungal cells, including yeast cells, and plant cells. Preferably the host cell is a microorganism. More preferably the microorganism belongs to bacteria. The term bacteria includes both Gram-negative and Gram-positive microorganisms. Examples of Gram-negative bacteria are, for example, any from the genera Escherichia, Gluconobacter, Rhodobacter, Pseudomonas, and Paracoccus. Gram-positive bacteria are selected from, but not limited to any of the families Bacillaceae, Brevibacteriaceae, Corynebacteriaceae, Lactobacillaceae, and Streptococcaceae and belong especially to the genera Bacillus, Brevibacterium, Corynebacterium, Lactobacillus, Lactococcus and Streptomyces. Among the genus Bacillus, B. subtilis, B. amyloliquefaciens, B. licheniformis and B. pumilus are preferred microorganisms in the context of the present invention. Among Gluconobacter, Rhodobacter and Paracoccus genera G. oxydans, R. sphaeroides and P. zeaxanthinifaciens are preferred, respectively.

Examples of yeasts are Saccharomyces, particularly S. cerevisiae. Examples of other preferred fungi are Aspergillus niger and Penicillium chrysogenum.

Microorganisms which can be used in the present invention in order to improve the direct production of Hy-T may be publicly available from different sources, e.g., Deutsche Sammlung von Mikroorganismen and Zellkulturen (DSMZ), Mascheroder Weg 1B, D-38124 Braunschweig, Germany, American Type Culture Collection (ATCC), P.O. Box 1549, Manassas, Va. 20108 USA or Culture Collection Division, NITE Biological Resource Center, 2-5-8, Kazusakamatari, Kisarazu-shi, Chiba, 292-0818, Japan (formerly: Institute for Fermentation, Osaka (IFO), 17-85, Juso-honmachi 2-chome, Yodogawa-ku, Osaka 532-8686, Japan).

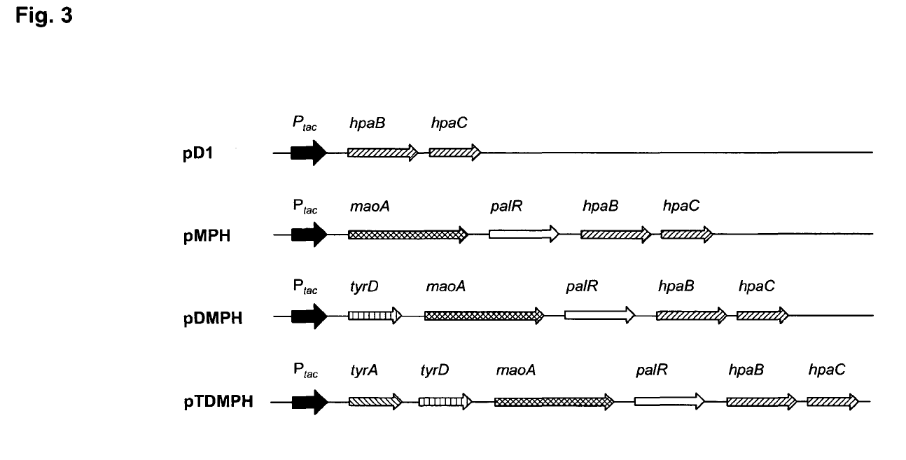

Preferred examples of microorganism according to the invention derive from the Escherichia coli K-12 strain TOP10, which is available from Invitrogen, and comprise plasmids as shown in FIG. 3.

In FIG. 3 all genes were inserted in the multiple cloning site (MCS) of cloning vector pJF119EH (Furste, J. P. et al., Gene (1986) 48: 119-131) which also carries the ampicillin resistance gene (bla): tyrA, chorismate mutase/prephenate dehydrogenase from E. coli MG1655; tyrD, L-tyrosine decarboxylase from Methanocaldococcus jannaschii; maoA, monoamine oxidase from E. coli MG1655; palR, phenylacetaldehyde reductase from Rhodococcus erythropolis (DSM 43297); HpaBC, 4-hydroxyphenylacetic acid 3-monooxygenase operon from E. coli W (ATCC 11105).

In particular, the present invention is related to a process for the direct production of Hy-T wherein at least one—preferably a combination—of polynucleotides or modified polynucleotides disclosed herein are introduced into a suitable microorganism, the recombinant microorganism is cultured under conditions that allow the production of Hy-T in high productivity, yield, and/or efficiency, the produced fermentation product is isolated from the culture medium and optionally further purified.

Several enzyme substrates may be used as starting material in the above-mentioned process. Compounds particularly suited as starting material are glucose, prephenate, L-tyrosine, L-phenylalanine, L-3,4-dihydroxyphenylalanine, 4-hydroxyphenylpyruvate, tyramine, 2-phenylethylamine, dopamine, phenylpyruvate, 4-hydroxyphenylacetaldehyde, phenylacetaldehyde, tyrosol, 2-(3-hydroxyphenyl)ethanol, phenylethanol or mixtures thereof.

Conversion of the substrate into Hy-T in connection with the above process using a microorganism means that the conversion of the substrate resulting in Hy-T is performed by the microorganism, i.e. the substrate may be directly converted into Hy-T. Said microorganism is cultured under conditions which allow such conversion from the substrate as defined above.

A medium as used herein for the above process using a microorganism may be any suitable medium for the production of Hy-T. Typically, the medium is an aqueous medium comprising for instance salts, substrate(s), and a certain pH. The medium in which the substrate is converted into Hy-T is also referred to as the production medium.

“Fermentation” or “production” or “fermentation process” or “biotransformation” or “bioconversion” or “conversion” as used herein may be the use of growing cells using any cultivation medium, conditions and procedures known to the skilled person, or the use of non-growing so-called resting cells, after they have been cultivated by using any growth medium, conditions and procedures known to the skilled person, under appropriate conditions for the conversion of suitable substrates into desired products such as Hy-T.

As used herein, resting cells refer to cells of a microorganism which are for instance viable but not actively growing due to omission of an essential nutrient from the medium, or which are growing at low specific growth rates [μ], for instance, growth rates that are lower than 0.02 h⁻¹, preferably lower than 0.01 h⁻¹. Cells which show the above growth rates are said to be in a “resting cell mode”. Microorganisms in resting cell mode may be used as cell suspensions in a liquid medium, be it aqueous, organic, or a mixture of aqueous and organic solvents; or as flocculated or immobilized cells on a solid phase, be it a porous or polymeric matrix.

The process of the present invention may be performed in different steps or phases. In one step, referred to as step (a) or growth phase, the microorganism can be cultured under conditions that enable its growth. In another step, also referred to as step (b) or transition phase, cultivation conditions can be modified so that the growth rate of the microorganism decreases until a resting cell mode is reached. In yet another step, also referred to as step (c) or production phase, Hy-T is produced from a substrate in the presence of the microorganism. In processes using resting cells, step (a) is typically followed by steps (b) and (c). In processes using growing cells, step (a) is typically followed by step (c).

Growth and production phases as performed in the above process using a microorganism may be performed in the same vessel, i.e., only one vessel, or in two or more different vessels, with an optional cell separation step between the two phases. The produced Hy-T can be recovered from the cells by any suitable means. Recovery means for instance that the produced Hy-T may be separated from the production medium. Optionally, the thus produced Hy-T may be further processed.

For the purpose of the present invention relating to the above process, the terms “growth phase”, “growing step”, “growth step” and “growth period” are used interchangeably herein. The same applies for the terms “production phase”, “production step”, “production period”.

One way of performing the above process may be a process wherein the microorganism is grown in a first vessel, the so-called growth vessel, as a source for the resting cells, and at least part of the cells are transferred to a second vessel, the so-called production vessel. The conditions in the production vessel may be such that the cells transferred from the growth vessel become resting cells as defined above. Hy-T is produced in the second vessel and recovered therefrom.

In connection with the above process, the growing step can be performed in an aqueous medium, i.e. the growth medium, supplemented with appropriate nutrients for growth under aerobic conditions. The cultivation may be conducted, for instance, in batch, fed-batch, semi-continuous or continuous mode. The cultivation period may vary depending on the kind of cells, pH, temperature and nutrient medium to be used, and may be for instance about 10 h to about 10 days, preferably about 1 to about 10 days, more preferably about 1 to about 5 days when run in batch or fed-batch mode, depending on the microorganism. If the cells are grown in continuous mode, the residence time may be for instance from about 2 to about 100 h, preferably from about 2 to about 50 h, depending on the microorganism. If the microorganism is selected from bacteria, the cultivation may be conducted for instance at a pH of about 3.0 to about 9.0, preferably about 4.0 to about 9.0, more preferably about 4.0 to about 8.0, even more preferably about 5.0 to about 8.0. If algae or yeast are used, the cultivation may be conducted, for instance, at a pH below about 7.0, preferably below about 6.0, more preferably below about 5.5, and most preferably below about 5.0. A suitable temperature range for carrying out the cultivation using bacteria may be for instance from about 13° C. to about 40° C., preferably from about 18° C. to about 37° C., more preferably from about 13° C. to about 36° C., and most preferably from about 18° C. to about 33° C. If algae or yeast are used, a suitable temperature range for carrying out the cultivation may be for instance from about 15° C. to about 40° C., preferably from about 20° C. to about 45° C., more preferably from about 25° C. to about 40° C., even more preferably from about 25° C. to about 38° C., and most preferably from about 30° C. to about 38° C. The culture medium for growth usually may contain such nutrients as assimilable carbon sources, e.g., glycerol, D-mannitol, D-sorbitol, L-sorbose, erythritol, ribitol, xylitol, arabitol, inositol, dulcitol, D-ribose, D-fructose, D-glucose, and sucrose, preferably L-sorbose, D-glucose, D-sorbitol, D-mannitol, and glycerol; and digestible nitrogen sources such as organic substances, e.g., peptone, yeast extract and amino acids. The media may be with or without urea and/or corn steep liquor and/or baker's yeast. Various inorganic substances may also be used as nitrogen sources, e.g., nitrates and ammonium salts. Furthermore, the growth medium usually may contain inorganic salts, e.g., magnesium sulfate, manganese sulfate, cupric sulfate, potassium phosphate, sodium phosphate, and calcium carbonate.

In connection with the above process, the specific growth rates are for instance at least 0.02 h⁻¹. For cells growing in batch, fed-batch or semi-continuous mode, the growth rate depends on for instance the composition of the growth medium, pH, temperature, and the like. In general, the growth rates may be for instance in a range from about 0.05 to about 0.2 h⁻¹, preferably from about 0.06 to about 0.15 h⁻¹, and most preferably from about 0.07 to about 0.13 h⁻¹.

In another aspect of the above process, resting cells may be provided by cultivation of the respective microorganism on agar plates thus serving as growth vessel, using essentially the same conditions, e.g., cultivation period, pH, temperature, nutrient medium as described above, with the addition of agar.

If the growth and production phase are performed in two separate vessels, then the cells from the growth phase may be harvested or concentrated and transferred to a second vessel, the so-called production vessel. This vessel may contain an aqueous medium supplemented with any applicable production substrate that can be converted to Hy-T by the cells. Cells from the growth vessel can be harvested or concentrated by any suitable operation, such as for instance centrifugation, membrane crossflow ultrafiltration or microfiltration, filtration, decantation, flocculation. The cells thus obtained may also be transferred to the production vessel in the form of the original broth from the growth vessel, without being harvested, concentrated or washed, i.e. in the form of a cell suspension. In a preferred embodiment, the cells are transferred from the growth vessel to the production vessel in the form of a cell suspension without any washing or isolation step in between.

If the growth and production phase are performed in the same vessel, cells may be grown under appropriate conditions to the desired cell density followed by a replacement of the growth medium with the production medium containing the production substrate. Such replacement may be, for instance, the feeding of production medium to the vessel at the same time and rate as the withdrawal or harvesting of supernatant from the vessel. To keep the resting cells in the vessel, operations for cell recycling or retention may be used, such as for instance cell recycling steps. Such recycling steps, for instance, include but are not limited to methods using centrifuges, filters, membrane crossflow microfiltration or ultrafiltration steps, membrane reactors, flocculation, or cell immobilization in appropriate porous, non-porous or polymeric matrixes. After a transition phase, the vessel is brought to process conditions under which the cells are in a resting cell mode as defined above, and the production substrate is efficiently converted into Hy-T.

Alternatively the cells could be used to produce Hy-T in growing mode such as when partially transforming a given substrate into Hy-T while partially using it as carbon source. Cells can be used as growing cells by supplying a carbon source and a substrate to be transformed into Hy-T or combinations of these. Cells can also be altered to be able to express the required activities upon induction by addition of external organic compounds (inducers).

The aqueous medium in the production vessel as used for the production step in connection with the above process using a microorganism, hereinafter called production medium, may contain only the production substrate(s) to be converted into Hy-T, or may contain for instance additional inorganic salts, e.g., sodium chloride, calcium chloride, magnesium sulfate, manganese sulfate, potassium phosphate, sodium phosphate, calcium phosphate, and calcium carbonate. The production medium may also contain digestible nitrogen sources such as for instance organic substances, e.g., peptone, yeast extract, urea, amino acids, and corn steep liquor, and inorganic substances, e.g. ammonia, ammonium sulfate, and sodium nitrate, at such concentrations that the cells are kept in a resting cell mode as defined above. The medium may be with or without urea and/or corn steep liquor and/or baker's yeast. The production step may be conducted for instance in batch, fed-batch, semi-continuous or continuous mode. In case of fed-batch, semi-continuous or continuous mode, both cells from the growth vessel and production medium can be fed continuously or intermittently to the production vessel at appropriate feed rates. Alternatively, only production medium may be fed continuously or intermittently to the production vessel, while the cells coming from the growth vessel are transferred at once to the production vessel. The cells coming from the growth vessel may be used as a cell suspension within the production vessel or may be used as for instance flocculated or immobilized cells in any solid phase such as porous or polymeric matrixes. The production period, defined as the period elapsed between the entrance of the substrate into the production vessel and the harvest of the supernatant containing Hy-T, the so-called harvest stream, can vary depending for instance on the kind and concentration of cells, pH, temperature and nutrient medium to be used, and is preferably about 2 to about 100 h. The pH and temperature can be different from the pH and temperature of the growth step, but is essentially the same as for the growth step.

In one embodiment, the production step is conducted in continuous mode, meaning that a first feed stream containing the cells from the growth vessel and a second feed stream containing the substrate is fed continuously or intermittently to the production vessel. The first stream may either contain only the cells isolated/separated from the growth medium or a cell suspension, coming directly from the growth step, i.e. cells suspended in growth medium, without any intermediate step of cell separation, washing and/or isolation and/or concentration. The second feed stream as herein defined may include all other feed streams necessary for the operation of the production step, e.g. the production medium comprising the substrate in the form of one or several different streams, water for dilution, and acid or base for pH control.

In connection with the above process, when both streams are fed continuously, the ratio of the feed rate of the first stream to feed rate of the second stream may vary between about 0.01 and about 10, preferably between about 0.01 and about 5, most preferably between about 0.02 and about 2. This ratio is dependent on the concentration of cells and substrate in the first and second stream, respectively.

Another way of performing the process as above using a microorganism of the present invention may be a process using a certain cell density of resting cells in the production vessel. The cell density is measured as absorbance units (optical density) at 600 nm by methods known to the skilled person. In a preferred embodiment, the cell density in the production step is at least about 2, more preferably between about 2 and about 200, even more preferably between about 10 and about 200, even more preferably between about 15 and about 200, even more preferably between about 15 to about 120, and most preferably between about 20 and about 120.

In order to keep the cells in the production vessel at the desired cell density during the production phase as performed, for instance, in continuous or semi-continuous mode, any means known in the art may be used, such as for instance cell recycling by centrifugation, filtration, membrane crossflow ultrafiltration or microfiltration, decantation, flocculation, cell retention in the vessel by membrane devices or cell immobilization. Further, in case the production step is performed in continuous or semi-continuous mode and cells are continuously or intermittently fed from the growth vessel, the cell density in the production vessel may be kept at a constant level by, for instance, harvesting an amount of cells from the production vessel corresponding to the amount of cells being fed from the growth vessel.

In connection with the above process, the produced Hy-T contained in the so-called harvest stream is recovered/harvested from the production vessel. The harvest stream may include, for instance, cell-free or cell-containing aqueous solution coming from the production vessel, which contains Hy-T as a result of the conversion of production substrate by the resting cells in the production vessel. Cells still present in the harvest stream may be separated from the Hy-T by any operations known in the art, such as for instance filtration, centrifugation, decantation, membrane crossflow ultrafiltration or microfiltration, tangential flow ultrafiltration or microfiltration or dead end filtration. After this cell separation operation, the harvest stream is essentially free of cells.

In a further aspect, the process of the present invention may be combined with further steps of separation and/or purification of the produced Hy-T from other components contained in the harvest stream, i.e., so-called downstream processing steps. These steps may include any means known to a skilled person, such as, for instance, concentration, extraction, crystallization, precipitation, adsorption, ion exchange, chromatography, distillation, electrodialysis, bipolar membrane electrodialysis and/or reverse osmosis. Any of these procedures alone or in combination constitute a convenient means for isolating and purifying the product, i.e. Hy-T. The product thus obtained may further be isolated in a manner such as, e.g. by concentration, crystallization, precipitation, washing and drying and/or further purified by, for instance, treatment with activated carbon, ion exchange and/or re-crystallization.

According to the invention, host cells that are altered to contain one or more genes capable of expressing an activity selected from the group defined above and exemplified herein are able to directly produce Hy-T from a suitable substrate in significantly higher yield, productivity, and/or efficiency than other known organisms.

Polynucleotides encoding enzymes as defined above and the selection thereof are hereinafter described in more detail. The term “gene” as used herein means a polynucleotide encoding a protein as defined above.

The invention encompasses polynucleotides as shown in SEQ ID NO: 1, SEQ ID NO: 3, SEQ ID NO:5, SEQ ID NO:7, SEQ ID NO:9, SEQ ID NO:11, SEQ ID NO:13, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32. SEQ ID NO:34, SEQ ID NO:36, SEQ ID NO:38, SEQ ID NO:40 and SEQ ID NO:42.

The invention also encompasses polynucleotides which are substantially homologous to one of these sequences. In this context it should be mentioned that the expression of “a polynucleotide which is substantially homologous” refers to a polynucleotide sequence selected from the group consisting of:
a) polynucleotides encoding a protein comprising the amino acid sequence according to SEQ ID NO:2, SEQ ID NO:4, SEQ ID NO:6, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41 and SEQ ID NO:43;
b) polynucleotides encoding a fragment or derivative of a polypeptide encoded by a polynucleotide of any of (a) wherein in said derivative one or more amino acid residues are conservatively substituted compared to said polypeptide, and said fragment or derivative has the activity of a HP or FG protein;
c) polynucleotides the complementary strand of which hybridizes under stringent conditions to a polynucleotide as defined in any one of (a) or (b) and which encode a HP or FG protein;
d) polynucleotides which are at least 70%, such as 85, 90 or 95% homologous to a polynucleotide as defined in any one of (a) to (c) and which encode a HP or FG polypeptide;
e) the complementary strand of a polynucleotide as defined in (a) to (d).

The invention also encompasses polypeptides as shown in SEQ ID NO: 2, SEQ ID NO: 4, SEQ ID NO:6, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33., SEQ ID NO:35, SEQ ID NO:37, SEQ ID NO:39, SEQ ID NO:41 and SEQ ID NO:43.

The invention also encompasses polypeptides which are substantially homologous to one of these amino acid sequences. In this context it should be mentioned that the expression of “a polypeptide which is substantially homologous” refers to a polypeptide sequence selected from the group consisting of:
a) polypeptides comprising an amino acid sequence comprising a fragment or derivative of a polypeptide sequence according to SEQ ID NO: 2, SEQ ID NO: 4, SEQ ID NO:6, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33; SEQ ID NO:35, SEQ ID NO:37, SEQ ID NO:39, SEQ ID NO:41 and SEQ ID NO:43, and which have the activity of a HP or FG polypeptide;
b) polypeptides comprising an amino acid sequence encoded by a fragment or derivative of a polynucleotide sequence according to SEQ ID NO: 1, SEQ ID NO: 3, SEQ ID NO:5, SEQ ID NO:7, SEQ ID NO:9, SEQ ID NO:11, SEQ ID NO:13, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32; SEQ ID NO:34, SEQ ID NO:36, SEQ ID NO:38, SEQ ID NO:40 and SEQ ID NO:42, and which have the activity of a HP or FG polypeptide;
c) polypeptides which are at least 50%, such as 70, 80 or 90% homologous to a polypeptide according to SEQ ID NO: 2, SEQ ID NO: 4, SEQ ID NO:6, SEQ ID NO:8, SEQ ID NO:10, SEQ ID NO:12, SEQ ID NO:14, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33; SEQ ID NO:35, SEQ ID NO:37, SEQ ID NO:39, SEQ ID NO:41 and SEQ ID NO:43, or to a polypeptide according to (a) or (b) and which have the activity of a HP or FG polypeptide.

An “isolated nucleic acid fragment” is a nucleic acid fragment that is not naturally occurring as a fragment and would not be found in the natural state.

As used herein, the terms “polynucleotide”, “gene” and “recombinant gene” refer to nucleic acid molecules which may be isolated from chromosomal or plasmid DNA or may be generated by synthetic methods, which include an open reading frame (ORF) encoding a protein as exemplified above. A polynucleotide may include a polynucleotide sequence or fragments thereof and regions upstream and downstream of the gene sequences which may include, for example, promoter regions, regulator regions and terminator regions important for the appropriate expression and stabilization of the polypeptide derived thereof.

A gene may include coding sequences, non-coding sequences such as for instance untranslated sequences located at the 3′- and 5′-ends of the coding region of a gene, and regulatory sequences. Moreover, a gene refers to an isolated nucleic acid molecule as defined herein. It is furthermore appreciated by the skilled person that DNA sequence polymorphisms that lead to changes in the amino acid sequences of the protein may exist within a gene population. Such genetic polymorphism in the gene may exist among individuals within a population due to natural variation or in cells from different populations. Such natural variations can typically result in 1-5% variance in the nucleotide sequence of the corresponding gene. Any and all such nucleotide variations and the resulting amino acid polymorphism are the result of natural variation. They do not alter the functional activity of proteins and therefore they are intended to be within the scope of the invention.

As used herein, the terms “polynucleotide” or “nucleic acid molecule” are intended to include DNA molecules (e.g., cDNA or genomic DNA) and RNA molecules (e.g., mRNA) and analogs of the DNA or RNA generated using nucleotide analogs. The nucleic acid molecule may be single-stranded or double-stranded, but preferably is double-stranded DNA. The nucleic acid may be synthesized using oligonucleotide analogs or derivatives (e.g., inosine or phosphorothioate nucleotides). Such oligonucleotides may be used, for example, to prepare nucleic acids that have altered base-pairing abilities or increased resistance to nucleases.

Unless otherwise indicated, all nucleotide sequences determined by sequencing a DNA molecule herein were determined using an automated DNA sequencer and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence may be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

The person skilled in the art is capable of identifying such erroneously identified bases and knows how to correct for such errors.

Homologous or substantially identical gene sequences may be isolated, for example, by performing PCR using two degenerate oligonucleotide primer pools designed on the basis of nucleotide sequences as taught herein.

The template for the reaction may be cDNA obtained by reverse transcription of mRNA prepared from strains known or suspected to express a polynucleotide according to the invention. The PCR product may be subcloned and sequenced to ensure that the amplified sequences represent the sequences of a new nucleic acid sequence as described herein, or a functional equivalent thereof.

The PCR fragment may then be used to isolate a full length cDNA clone by a variety of known methods. For example, the amplified fragment may be labeled and used to screen a bacteriophage or cosmid cDNA library. Alternatively, the labeled fragment may be used to screen a genomic library.

PCR technology can also be used to isolate full-length cDNA sequences from other organisms. For example, RNA may be isolated, following standard procedures, from an appropriate cellular or tissue source. A reverse transcription reaction may be performed on the RNA using an oligonucleotide primer specific for the most 5′-end of the amplified fragment for the priming of first strand synthesis.

The resulting RNA/DNA hybrid may then be “tailed” (e.g., with guanines) using a standard terminal transferase reaction, the hybrid may be digested with RNaseH, and second strand synthesis may then be primed (e.g., with a poly-C primer). Thus, cDNA sequences upstream of the amplified fragment may easily be isolated. For a review of useful cloning strategies, see e.g., Sambrook, et al. (Sambrook J. et al. “Molecular Cloning: A Laboratory Manual” Cold Spring Harbor (N.Y., USA): Cold Spring Harbor Laboratory Press, 2001); and Ausubel et al. (Ausubel F. M. et al., “Current Protocols in Molecular Biology”, John Wiley & Sons (N.Y., USA): John Wiley & Sons, 2007).

Homologues, substantially identical sequences, functional equivalents, and orthologs of genes and proteins exemplified herein, such as for example the gene according to SEQ ID NO:5, and the encoded protein according to SEQ ID NO:6, may be obtained from a number of different microorganisms. In this context it should be mentioned that also the following paragraphs apply mutatis mutandis for all other enzymes defined above.

The procedures for the isolation of specific genes and/or fragments thereof are exemplified herein. Accordingly, nucleic acids encoding other family members, which thus have a nucleotide sequence that differs from a nucleotide sequence according to SEQ ID NO:5, are within the scope of the invention. Moreover, nucleic acids encoding proteins from different species which thus have a nucleotide sequence which differs from a nucleotide sequence shown in SEQ ID NO:5 are within the scope of the invention.

The invention also discloses an isolated polynucleotide hybridisable under stringent conditions, preferably under highly stringent conditions, to a polynucleotide according to the present invention, such as for instance a polynucleotide shown in SEQ ID NO:5 Advantageously, such polynucleotide may be obtained from a microorganism capable of converting a given carbon source directly into Hy-T.

As used herein, the term “hybridizing” is intended to describe conditions for hybridization and washing under which nucleotide sequences at least about 50%, at least about 60%, at least about 70%, more preferably at least about 80%, even more preferably at least about 85% to 90%, most preferably at least 95% homologous to each other typically remain hybridized to each other.

A preferred, non-limiting example of such hybridization conditions are hybridization in 6× sodium chloride/sodium citrate (SSC) at about 45° C., followed by one or more washes in 1×SSC, 0.1% SDS at 50° C., preferably at 55° C., more preferably at 60° C. and even more preferably at 65° C.

Highly stringent conditions include, for example, 2 h to 4 days incubation at 42° C. using a digoxigenin (DIG)-labeled DNA probe (prepared by using a DIG labeling system; Roche Diagnostics GmbH, 68298 Mannheim, Germany) in a solution such as DigEasyHyb solution (Roche Diagnostics GmbH) with or without 100 μg/ml salmon sperm DNA, or a solution comprising 50% formamide, 5×SSC (150 mM NaCl, 15 mM trisodium citrate), 0.02% sodium dodecyl sulfate, 0.1% N-lauroylsarcosine, and 2% blocking reagent (Roche Diagnostics GmbH), followed by washing the filters twice for 5 to 15 minutes in 2×SSC and 0.1% SDS at room temperature and then washing twice for 15-30 minutes in 0.5×SSC and 0.1% SDS or 0.1×SSC and 0.1% SDS at 65-68° C.

The skilled artisan will know which conditions to apply for stringent and highly stringent hybridization conditions. Additional guidance regarding such conditions is readily available in the art, for example, in Sambrook et al., (supra), Ausubel et al. (supra). Of course, a polynucleotide which hybridizes only to a poly (A) sequence (such as the 3′-terminal poly (A) tract of mRNAs), or to a complementary stretch of T (or U) residues, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A nucleic acid molecule of the present invention, such as for instance a nucleic acid molecule shown in SEQ ID NO:5 or a fragment or derivative thereof, may be isolated using standard molecular biology techniques and the sequence information provided herein. For example, using all or portion of the nucleic acid sequence shown in SEQ ID NO:5 as a hybridization probe, nucleic acid molecules according to the invention may be isolated using standard hybridization and cloning techniques (e.g., as described in Sambrook et al. (supra)).

Furthermore, oligonucleotides corresponding to or hybridisable to nucleotide sequences according to the invention may be prepared by standard synthetic techniques, e.g., using an automated DNA synthesizer, or delivered by gene synthesis as carried out by companies such as, for example, DNA2.0 (DNA2.0, Menlo Park, 94025 CA, USA) based on the sequence information provided herein.

The terms “homology”, “identically”, “percent identity” or “similar” are used interchangeably herein. For the purpose of this invention, it is defined here that in order to determine the percent identity of two amino acid sequences or of two nucleic acid sequences, the sequences are aligned for optimal comparison purposes (e.g., gaps may be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length.

The skilled person will be aware of the fact that several different computer programs are available to determine the homology between two sequences. For instance, a comparison of sequences and determination of percent identity between two sequences may be accomplished using a mathematical algorithm. In a preferred embodiment, the percent identity between two amino acid sequences is determined using the Needleman and Wunsch algorithm (Needleman and Wunsch, J. Mol. Biol. (1970) 48:443-453) which has been incorporated into the GAP program in the GCG software package (available at http://www.accelrys.com), using either a BLOSUM62 matrix or a PAM250 matrix, and a gap weight of 16, 14, 12, 10, 8, 6 or 4 and a length weight of 1, 2, 3, 4, 5 or 6. The skilled person will appreciate that all these different parameters will yield slightly different results but that the overall percentage identity of two sequences is not significantly altered when using different algorithms.

In yet another embodiment, the percent identity between two nucleotide sequences is determined using the GAP program in the GCG software package (available at http://www.accelrys.com), using a NWSGAPDNA.CMP matrix and a gap weight of 40, 50, 60, 70 or 80 and a length weight of 1, 2, 3, 4, 5 or 6. In another embodiment, the percent identity between two amino acid or nucleotide sequences is determined using the algorithm of E. Meyers and W. Miller (Meyers and Miller, Comput. Appl. Biosci. (1989) 4:11-17) which has been incorporated into the ALIGN program (version 2.0) (available at http://vega.igh.cnrs.fr/bin/align-guess.cgi) using a PAM120 weight residue table, a gap length penalty of 12 and a gap penalty of 4.

The nucleic acid and protein sequences of the present invention may further be used as a “query sequence” to perform a search against public databases to, for example, identify other family members or related sequences. Such searches may be performed using the BLASTN and BLASTP programs (version 2.0) of Altschul, et al. (J. Mol. Biol. (1990) 215:403-410). BLAST nucleotide searches may be performed with the BLASTN program, score=100, word length=12 to obtain nucleotide sequences homologous to the nucleic acid molecules of the present invention. BLAST protein searches may be performed with the BLASTP program, score=50, word length=3 to obtain amino acid sequences homologous to the protein molecules of the present invention. To obtain gapped alignments for comparison purposes, Gapped BLAST may be utilized as described in Altschul et al., (Nucleic Acids Res. (1997) 25:3389-3402). When utilizing BLAST and Gapped BLAST programs, the default parameters of the respective programs (e.g., BLASTP and BLASTN) may be used (see for example http://www.ncbi.nim.nih.gov.)

In another embodiment, an isolated nucleic acid molecule of the invention comprises a nucleic acid molecule which is the complement of a nucleotide sequence as of the present invention, such as for instance the sequence shown in SEQ ID NO:5. A nucleic acid molecule, which is complementary to a nucleotide sequence disclosed herein, is one that is sufficiently complementary to a nucleotide sequence shown in SEQ ID NO:5 such that it may hybridize to said nucleotide sequence thereby forming a stable duplex.

In a further embodiment, a nucleic acid of the invention, as for example shown in SEQ ID NO:5, or the complement thereof contains at least one mutation leading to a gene product with modified function/activity. The at least one mutation may be introduced by methods known in the art or described herein. In regard to the group of enzymes exemplified herein above, the at least one mutation leads to a protein whose function compared to the wild type counterpart is enhanced or improved. The activity of the protein is thereby increased. Methods for introducing such mutations are well known in the art.

Another aspect pertains to vectors, containing a nucleic acid encoding a protein according to the invention or a functional equivalent or portion thereof. As used herein, the term “vector” refers to a nucleic acid molecule capable of transporting another nucleic acid to which it has been linked. One type of vector is a “plasmid”, which refers to a circular double stranded DNA molecule into which additional DNA segments may be incorporated. Another type of vector is a viral vector, wherein additional DNA segments may be inserted into the viral genome. Certain vectors are capable of autonomous replication in a host cell into which they are introduced (e.g., bacterial vectors having an origin of DNA replication that is functional in said bacteria). Other vectors are integrated into the genome of a host cell upon introduction into the host cell, and thereby are replicated along with the host genome.

Moreover, certain vectors are capable of directing the expression of genes to which they are operatively linked. Such vectors are referred to herein as “expression vectors”. In general, expression vectors of utility in recombinant DNA techniques are often in the form of plasmids. The terms “plasmid” and “vector” can be used interchangeably herein as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention may be designed for expression of enzymes as defined above in a suitable microorganism. Expression vectors useful in the present invention include chromosomal-, episomal- and virus-derived vectors e.g., vectors derived from bacterial plasmids, bacteriophage, and vectors derived from combinations thereof, such as those derived from plasmid and bacteriophage genetic elements, such as cosmids and phagemids.

The recombinant vectors of the invention comprise a nucleic acid of the invention in a form suitable for expression of the nucleic acid in a host cell, which means that the recombinant expression vector includes one or more regulatory sequences, selected on the basis of the host cells to be used for expression, which is operatively linked to the nucleic acid sequence to be expressed. Within a recombinant expression vector, “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., attenuators). Such regulatory sequences are described, for example, in “Methods in Enzymology”, Volume 185: “Gene Expression Technology”, Goeddel DV (Ed.), Academic Press (San Diego, Calif.), 1990. Regulatory sequences include those which direct constitutive or inducible expression of a nucleotide sequence in many types of host cells and those which direct expression of the nucleotide sequence only in a certain host cell (e.g. tissue-specific regulatory sequences). It will be appreciated by those skilled in the art that the design of the expression vector can depend on such factors as the choice of the host cell to be transformed, the level of expression of protein desired, etc. The expression vectors of the invention may be introduced into host cells to thereby produce proteins or peptides, encoded by nucleic acids as described herein, including, but not limited to, mutant proteins, fragments thereof, variants or functional equivalents thereof, and fusion proteins, encoded by a nucleic acid as described herein.

The DNA insert may be operatively linked to an appropriate promoter, which may be either a constitutive or inducible promoter. The skilled person will know how to select suitable promoters. The expression constructs may contain sites for transcription initiation, termination, and, in the transcribed region, a ribosome binding site for translation. The coding portion of the mature transcripts expressed by the constructs may preferably include an initiation codon at the beginning and a termination codon appropriately positioned at the end of the polypeptide to be translated.

Vector DNA may be introduced into suitable host cells via conventional transformation or transfection techniques. As used herein, the terms “transformation”, “conjugation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, transduction, infection, lipofection, cationic lipid-mediated transfection or electroporation. Suitable methods for transforming or transfecting host cells may be found in Sambrook, et al. (supra), Davis et al., (“Basic Methods in Molecular Biology”, Elsevier (N.Y., USA), 1986) and other laboratory manuals.

In order to identify and select cells which have integrated the foreign DNA into their genome, a gene that encodes a selectable marker (e.g., resistance to antibiotics) is generally introduced into the host cells along with the gene of interest. Preferred selectable markers include those that confer resistance to drugs, such as kanamycin, tetracycline, ampicillin and streptomycin. A nucleic acid encoding a selectable marker is preferably introduced into a host cell on the same vector as that encoding a protein according to the invention or can be introduced on a separate vector such as, for example, a suicide vector, which cannot replicate in the host cells. Cells stably transfected with the introduced nucleic acid can be identified by drug selection (e.g., cells that have incorporated the selectable marker gene will survive, while the other cells die).

As mentioned above, the polynucleotides of the present invention may be utilized in the genetic engineering of a suitable host cell to make it better and more efficient in the production, for example in a direct fermentation process, of Hy-T.

Therefore, the invention also relates to the concurrent use of genes encoding polypeptides having activities as specified above. Such a host cell will then show an improved capability to directly produce Hy-T.

The alteration in the genome of the microorganism may be obtained e.g. by replacing through a single or double crossover recombination a wild type DNA sequence by a DNA sequence containing the alteration. For convenient selection of transformants of the microorganism with the alteration in its genome the alteration may, e.g. be a DNA sequence encoding an antibiotic resistance marker or a gene complementing a possible auxotrophy of the microorganism. Mutations include, but are not limited to, deletion-insertion mutations.

An alteration in the genome of the microorganism leading to a more functional polypeptide may also be obtained by randomly mutagenizing the genome of the microorganism using e.g. chemical mutagens, radiation or transposons and selecting or screening for mutants which are better or more efficient producers of one or more fermentation products. Standard methods for screening and selection are known to the skilled person.

In another specific embodiment, it is desired to enhance and/or improve the activity of a protein selected from the group of enzymes specified herein above.

The invention also relates to microorganisms wherein the activity of a given polypeptide is enhanced and/or improved so that the yield of Hy-T which is directly produced is increased, preferably in those organisms that overexpress the said polypeptides or an active fragment or derivative thereof. This may be accomplished, for example, by transferring a polynucleotide according to the invention into a recombinant or non-recombinant microorganism that may or may not contain an endogenous equivalent of the corresponding gene.

The skilled person will know how to enhance and/or improve the activity of a protein. Such may be accomplished by either genetically modifying the host organism in such a way that it produces more or more stable copies of the said protein than the wild type organism. It may also be accomplished by increasing the specific activity of the protein.

In the following paragraphs procedures are described how to achieve this goal, i.e. the increase in the yield and/or production of Hy-T by increasing (up-regulation) the activity of a specific protein. These procedures apply mutatis mutandis for the similar proteins whose functions, compared to the wild type counterpart, have to be enhanced or improved.

Modifications in order to have the organism produce more copies of specific gene, i.e. overexpressing the gene, and/or protein may include the use of a strong promoter, or the mutation (e.g. insertion, deletion or point mutation) of (parts of) the gene or its regulatory elements. It may also involve the insertion of multiple copies of the gene into a suitable microorganism. An increase in the specific activity of a protein may also be accomplished by methods known in the art. Such methods may include the mutation (e.g. insertion, deletion or point mutation) of (parts of) the encoding gene.

A mutation as used herein may be any mutation leading to a more functional or more stable polypeptide, e.g. more functional or more stable gene products. This may include for instance an alteration in the genome of a microorganism, which improves the synthesis of the protein or leads to the expression of the protein with an altered amino acid sequence whose function compared with the wild type counterpart having a non-altered amino acid sequence is improved and/or enhanced. The interference may occur at the transcriptional, translational or post-translational level.

The term “increase” of activity as used herein encompasses increasing activity of one or more polypeptides in the producing organism, which in turn are encoded by the corresponding polynucleotides described herein. There are a number of methods available in the art to accomplish the increase of activity of a given protein. In general, the specific activity of a protein may be increased or the copy number of the protein may be increased.

To facilitate such an increase, the copy number of the genes corresponding to the polynucleotides described herein may be increased. Alternatively, a strong promoter may be used to direct the expression of the polynucleotide. In another embodiment, the promoter, regulatory region and/or the ribosome binding site upstream of the gene can be altered to increase the expression. The expression may also be enhanced or increased by increasing the relative half-life of the messenger RNA. In another embodiment, the activity of the polypeptide itself may be increased by employing one or more mutations in the polypeptide amino acid sequence, which increases the activity. For example, lowering the relative Km and/or increasing the kcat of the polypeptide with its corresponding substrate will result in improved activity. Likewise, the relative half-life of the polypeptide may be increased. In either scenario, that being enhanced gene expression or increased specific activity, the improvement may be achieved by altering the composition of the cell culture medium and/or methods used for culturing. “Enhanced expression” or “improved activity” as used herein means an increase of at least 5%, 10%, 25%, 50%, 75%, 100%, 200% or even more than 500%, compared to a wild-type protein, polynucleotide, gene; or the activity and/or the concentration of the protein present before the polynucleotides or polypeptides are enhanced and/or improved. The activity of the protein may also be enhanced by contacting the protein with a specific or general enhancer of its activity.

The invention is further illustrated by the following examples which should not be construed as limiting.

### Materials and Methods

### Strains and Plasmids

Bacterial strains used for the invention were Escherichia coli W (ATCC 11105, American Type Culture Collection), Escherichia coli DH10B, Escherichia coli TOP10 (Invitrogen), Escherichia coli MG1655 (CGSC No. 7740, E. coli Genetic Stock Center), Acinetobacter calcoaceticus EBF 65/61 (Barrowman M. M. and Fewson C. A. Curr. Microbiol. (1985) 12:235-240), Pseudomonas putida U, Pseudomonas putida A7 (Olivera E. R. et al. Eur. J. Biochem. (1994) 221:375-381), Pseudomonas putida KT2440 (DSMZ 6125, German Collection of Microorganisms and Cell Cultures), Rhodococcus erythropolis (DSMZ 43297, German Collection of Microorganisms and Cell Cultures). Plasmids used in this study were pCR-XL-TOPO (Invitrogen), pZErO-2 (Invitrogen), pCK01, pUC18, and pJF119EH (Furste et al., Gene (1986) 48: 119-131) and pJF119EH hpaB hpaC (also referred to as pJF hpaB hpaC, pJFhpaBC, or pD1). Plasmid pJF119EH hpaB hpaC (alias pD1) is described in WO 2004/015094 and was deposited under the Budapest Treaty on 23 Jul. 2002 with the DSMZ under number DSM 15109.

TABLE 1
Description of strains and plasmids used for hydroxytyrosol production

| Host Strain & | |
|---|---|
| Plasmids | Description |
| E. coli TOP10 | F⁻ mcrA Δ(mrr⁻hsdRMS⁻mcrBC) φ80lacZΔM15 ΔlacX74 deoR recA1 endA1 |
| | araΔ139 Δ(ara, leu)7697 galU galKλ- rpsL(StrR) nupG. |
| pD1 = pJFhpaBC | hpaBC genes coding for 4-hydroxyphenylacetic acid 3-monooxygenase from |
| | E. coli W ATCC 11105 cloned as a BamHI/HindIII fragment in the MCS of |
| | vector pJF119EH under the control of an IPTG-inducible tac promoter; Ap^{R}. |
| pPH | palR ORF coding for phenylacetaldehyde reductase from Rhodococcus |
| | erythropolis (DSMZ 43297) cloned as a SmaI/BamHI fragment in plasmid |
| | pD1 under the control of an IPTG-inducible tac promoter; Ap^{R}. |
| pMPH | maoA ORF coding for monoamine oxidase from E. coli MG1655 (CGSC # |
| | 7740) cloned as a EcoRI/SmaI fragment in in plasmid pPH under the control |
| | of an IPTG-inducible tac promoter; Ap^{R}. |
| pDMPH | tyrD codon optimized synthetic gene (DNA 2.0) coding for L-tyrosine |
| | decarboxylase from Methanocaldococcus jannaschii cloned as a EcoRI/KpnI |
| | fragment in plasmid pMPH under the control of an IPTG-inducible tac |
| | promoter; Ap^{R}. |
| pTDMPH | tyrA coding for chorismate mutase/prephenate dehydrogenase from E. coli |
| | MG1655 (CGSC # 7740) cloned as a EcoRI/EcoRI fragment in in plasmid |
| | pDMPH under the control of an IPTG-inducible tac promoter; Ap^{R}. |

### General Microbiology

All solutions were prepared in deionized water. LB medium (1 L) contained Bacto tryptone (10 g), Bacto yeast extract (5 g), and NaCl (10 g). 2*TY medium (1 L) contained Bacto tryptone (16 g), Bacto yeast extract (10 g) and NaCl (5 g). Nutrient broth (1 L) contained peptone (5 g) and meat extract (3 g). M9 salts (1 L) contained Na₂HPO₄ (6 g), KH₂PO₄ (3 g), NH₄Cl (1 g), and NaCl (0.5 g). M9 medium contained D-glucose (4 g) and MgSO₄ (1 mM) in 1 L of M9 salts. M9 inoculation medium contained D-glucose (4 g), casamino acids (20 g) and MgSO₄ (1 mM) in 1 L of M9 salts. M9 induction medium contained D-glucose (40 g), casamino acids (20 g) and MgSO₄ (1 mM) in 1 L of M9 salts. Unless stated otherwise, antibiotics were added where appropriate to the following final concentrations: ampicillin (Ap), 100 mg/L; kanamycin (Km), 50 mg/L; chloramphenicol (Cm), 33 mg/L. Casamino acids (Difco cat. no. 223120) were prepared as 20% stock solution in water. Stock solutions of 4-hydroxyphenylacetic acid (405 mM), tyrosol (405 mM), tyramine (810 mM) were prepared in potassium phosphate buffer (50 mM, pH 7.0); L-tyrosine (0.2-0.3 M) was titrated into solution using KOH. Isopropyl-β-D-thiogalactopyranoside (IPTG) was prepared as a 100 mM stock solution in water. Solutions of LB medium, M9 salts, MgSO₄, and D-glucose were autoclaved individually prior to mixing. Copper(II) sulphate (CuSO₄) was prepared as a 50 mM stock solution in water and added to bacterial cells as specified in the text. Solutions of antibiotics, casamino acids, tyrosol, 4-hydroxyphenylacetic acid, tyramine, L-tyrosine, ascorbic acid, glycerol, IPTG and CuSO₄ were sterilized through 0.22-μm membranes. Solid medium was prepared by addition of Difco agar to a final concentration of 1.5% (w/v). Unless otherwise stated, liquid cultures of E. coli were grown at 37° C. with agitation at 250 rpm and solid cultures were incubated at 30° C. Bacterial growth was monitored by measuring the optical density (O.D.) of liquid cultures at 600 nm (OD₆₀₀) using a spectrophotometer. Standard molecular cloning techniques well known to those skilled in the art were performed for construction and analysis of plasmid DNA as well as for transformation of E. coli strains as described in Sambrook J. et al. “Molecular Cloning: A Laboratory Manual” Cold Spring Harbor (N.Y., USA): Cold Spring Harbor Laboratory Press, 2001. Commercially available kits for the isolation and amplification of nucleic acids were used according to manufacturer's instructions. QIAprep Spin Miniprep Kit was purchased from Qiagen and used for plasmid DNA isolation. High Pure PCR Template Preparation Kit was purchased from Roche Diagnostics and used for chromosomal DNA isolation. Polymerase chain reactions (PCR) were performed with Herculase™ Enhanced DNA Polymerase from Stratagene using iCycler, a thermal cycler from BioRad. Restriction enzymes were purchased from New England Biolabs or Roche Diagnostics. Nucleic acid ligations were performed using T4 ligase from Roche Diagnostics.

### Preparation of Working Cell Banks

Inoculants of E. coli strains were started by introducing one single colony picked off a freshly streaked agar plate into 5 mL of M9 inoculation medium containing the appropriate antibiotic. Cultures were grown for 24 h then used to inoculate 50 mL of M9 induction medium containing the appropriate antibiotic to a starting OD₆₀₀, of 0.025-0.05 (1% inoculum). The 50 mL culture was grown at 37° C. with agitation at 250 rpm to OD₆₀₀=0.4-0.6 then used to prepare several frozen cell stocks in 20% glycerol (up to 27 cryovials per culture). Typically, 0.75 mL cell suspension was aseptically mixed with 0.25 mL 80% glycerol then stocked at −80° C. until used.

### HPLC Analysis

Reactions were sampled (1.0 mL) at several time-points during the cultivation or incubation period. Samples were centrifuged to remove cells debris. The clear supernatant (0.75 mL) was transferred to an amber glass vial for HPLC analysis. Reverse phase HPLC methods were developed for the simultaneous quantification of tyrosol, hydroxytyrosol, 4-hydroxyphenylacetic acid, 3,4-dihydroxyphenylacetic acid, tyramine, L-tyrosine and related substances (see below): Method 2 results in a better resolution of L-tyrosine and tyramine compared to Method 1 (Table 2). HPLC was performed on an Agilent 1100 HPLC system equipped with a thermostatic autosampler and a diode array detector. The separation was carried out using a Phenomenex Security Guard C18 guard column (4 mm×3.0 mm I.D.) and a YMC Pack ProC18 analytical column (5 μm, 150 mm×4.6 mm ID.). The column temperature was maintained at 23° C. and the flow rate at 1.0 mL/min. Typically, the column pressure varied from 70 (at start) to 120 bar. Sample detection was achieved at 210 nm. The injection volume was 3 μL. Compounds were identified by comparison of retention times and their online-recorded UV spectra with those of reference compounds. Concentrations were calculated by integration of peak areas and based on previously constructed standard calibration curves (see Table 2 for list of retention times).

Method 1: a gradient of acetonitrile (ACN) in 0.1% aqueous methanesulfonic acid was used as a mobile phase with the following elution profile: 0 to 5 min, 10% ACN; 5 to 20 min, increase ACN to 90%; 20 to 25 min, hold ACN at 90%.

Method 2: a gradient of ACN in 0.1% aqueous methanesulfonic acid was used as a mobile phase with the following elution profile: 0 to 3 min, 6% ACN; 4 to 20 min, increase ACN to 70%; 20 to 25 min, hold ACN at 70%.

TABLE 2
HPLC retention times

| | | Retention Time (min) | |
|---|---|---|---|
| | Compound | Method 1 | Method 2 |
| Compound Name | Abbreviation | (old) | (new) |
| Dopamine | Dopa-NH2 | 1.75 | 2.12 |
| Tyramine | Tyr-NH2 | 2.03 | 2.50 |
| 1-Tyrosine | Tyr | 2.19 | 2.92 |
| 1-Phenylalanine | Phe | 3.25 | 5.10 |
| 2-Phenylethylamine | Phe-NH2 | 3.60 | 5.71 |
| Hydroxytyrosol | HO-Tyrosol | 4.80 | 7.65 |
| 3,4-Dihydroxyphenylacetic acid | 3,4-DHPA | 6.50 | 9.11 |
| Tyrosol | 4-HPE | 7.80 | 10.00 |
| 4-Hydroxyphenylacetic acid | 4-HPA | 9.59 | 11.35 |
| 2-(3-Hydroxyphenyl)ethanol | 3-HPE | 9.63 | 11.39 |
| 2-Phenylethanol | 2-PE | 12.7 | 13.29 |
| 4-Methoxyphenylacetic acid | 4-MEPA | 13.3 | 15.57 |

Construction of Plasmid pMPH

E. coli strain TOP10 (Invitrogen) was engineered to express genes encoding enzymatic activities that enable side-chain modification of tyramine via 4-hydroxyphenylaldehyde and via tyrosol to hydroxytyrosol.

The palR open reading frame (ORF) coding for phenylacetaldehyde reductase was amplified by PCR using Rhodococcus erythropolis (DSMZ 43297) chromosomal DNA as template, 5′CCCGGGTAAGGAGGTGATCAAATGAAGGCAATCCAGTACACG-3′ (SmaI restriction site is underlined, ribosome binding site (rbs) and palR start codon are in boldface) as the forward primer, and 5′-GGATCCCTACAGACCAGGGACCACAACCG-3′ (BamHI restriction site is underlined) as the reverse primer. PCR mixtures (50 μL) contained 0.5 mg R. erythropolis (DSMZ 43297) chromosomal DNA, 50 pmol of each primer, 12.5 nmol of each deoxynucleotide (dNTPs), 5 U of Herculase DNA polymerase (Stratagene). PCR amplification started with a first denaturation step (95° C. for 5 min) followed by 35 repeats of temperature cycling steps (94° C. for 45 s, 55° C. for 45 s, and 72° C. for 90 s). The 1.1-kb PCR product was analyzed and gel-purified by agarose gel electrophoresis then mixed with vector pCR-XL-TOPO according to the TOPO® XL PCR Cloning Kit protocol (Invitrogen) to yield plasmid pPalR, which was subjected to DNA sequence analysis. The palR ORF was excised from plasmid pPalR by digestion with SmaI and BamHI and the 1.1-kb DNA fragment ligated to SmaI/BamHI-digested plasmid pJFhpaBC with T4 DNA ligase at 16° C. for 16 h. Ligation mixtures were used to transform E. coli TOP10 competent cells. Ampicilling-resistant transformants were selected on LB solid medium and analyzed for palR insertion, which afforded plasmid pJF palR hpaBC (also referred to as pPH).

The maoA ORF coding for monoamine oxidase was amplified by PCR using Escherichia coli MG1655 (CGSC # 7740) chromosomal DNA as template, 5′-GAATTCGGTACCTAAGGAGGTGATCAAATGGGAAGCCCCTCTCTG-3′ (EcoRI and KpnI restriction site are underlined, ribosome binding site (rbs) and maoA start codon are in boldface) as the forward primer, and 5′CCCGGGTCACTTATCTTTCTTCAGCG-3′ (SmaI restriction site is underlined) as the reverse primer. PCR mixtures (50 μL) contained 0.5 mg E. coli MG1655 chromosomal DNA, 50 μmol of each primer, 12.5 nmol of each dNTPs, 5 U of Herculase DNA polymerase (Stratagene). PCR amplification started with a first denaturation step (95° C. for 5 min) followed by 35 repeats of temperature cycling steps (94° C. for 45 s, 55° C. for 45 s, and 72° C. for 150 s). The 2.0-kb PCR product was analyzed and gel-purified by agarose gel electrophoresis then mixed with vector pCR-XL-TOPO according to the TOPO® XL PCR Cloning Kit protocol (Invitrogen) to yield plasmid pMaoA, which was subjected to DNA sequence analysis. The maoA ORF was excised from plasmid pMaoA by digestion with EcoRI and SmaI and the 2.0-kb DNA fragment ligated to EcoRI/SmaI-digested plasmid pPH. Ligation mixtures were used to transform E. coli TOP10 competent cells. Ampicilling-resistant transformants were selected on LB solid medium and analyzed for maoA insertion, which afforded plasmid pJF maoA palR hpaBC (also referred to as pMPH).

Construction of Plasmid pDMPH.

Enzymatic activities that decarboxylate L-tyrosine to yield tyramine are well-characterized in eukaryotic organisms, especially in plants, but to a lesser extent in prokaryotes. Microorganisms responsible for the occurrence of tyramine at potentially hazardous concentrations in fermented foods and beverages were identified as belonging to the genera Lactobacillus, Leuconostoc, Lactococcus, Enterococcus, or Carnobacterium and shown to express L-tyrosine decarboxylase activity. The functional role of putative L-tyrosine decarboxylase genes was recently established in a few bacteria such as Enterococcus faecalis (Connil N. et al. Appl. Environ. Microbiol. (2002) 68:3537-3544), Lactobacillus brevis IOEB 9809 (Lucas P. et al. FEMS Microbiol. Lett (2003) 229:65-71), and Carnobacterium divergens 508 (Coton M. et al. Food Microbiol. (2004) 21:125-130). A functional L-phenylalanine/L-tyrosine decarboxylase from Enterococcus faecium RM58 was also genetically characterized (Marcobal A. et al. FEMS Microbiol. Lett. (2006) 258:144-149). Putative L-tyrosine decarboxylase genes were identified by homology searches in all complete methanoarcheal genome sequences and even characterized in Methanocaldococcus jannaschii (Kezmarsky N. D. et al. Biochim. Biophys. Acta (2005) 1722:175-182).

The tyrD ORF coding for L-tyrosine decarboxylase was made available by custom gene synthesis as carried out by DNA 2.0 Inc (USA) upon codon optimization of the mfnA gene from Methanocaldococcus jannaschii locus MJ0050 for improved heterologous protein expression in E. coli. The synthetic tyrD gene was received as an insert in plasmid pJ36:5867, from which it was excised by digestion with EcoRI and KpnI. The resulting 1.2-kb DNA fragment was ligated to EcoRI/KpnI-digested vector pUC18 to yield plasmid pUC tyrD (also referred to as pUCTD).

Digestion of plasmid pMPH with EcoRI and KpnI yielded two DNA fragments, 2.9-kb and 7.9-kb in size. The 1.2-kb tyrD locus was excised from plasmid pJ36:5867 by EcoRI and KpnI digestion and ligated to the gel-purified 7.9-kb DNA fragment from pMPH, yielding plasmid pJDAMP in which maoA and palR genes are disrupted. The smaller 2.9-kb DNA fragment, also gel-purified from EcoRI/KpnI-digested plasmid pMPH, was ligated to KpnI-digested plasmid pJDAMP to yield plasmid pJF tyrD maoA palR hpaBC (also referred to as pDMPH).

Construction of Plasmid pTDMPH

The tyrA ORF coding for chorismate mutase/prephenate dehydrogenase was amplified by PCR using Escherichia coli MG1655 (CGSC # 7740) chromosomal DNA as template, 5′-gcggccgcTAAGGAGGTgatcaaATGgttgctgaattgaccgc-3′ (NotI restriction site is underlined, ribosome binding site (rbs) and tyrA start codon are in boldface) as the forward primer, and 5′Ctcgagtctagattactggcgattgtcattcg-3′ (XhoI and XbaI restriction sites are underlined) as the reverse primer. PCR mixtures (50 μL) contained 0.5 mg E. coli MG1655 chromosomal DNA, 50 pmol of each primer, 12.5 nmol of each dNTPs, 5 U of Herculase DNA polymerase (Stratagene). PCR amplification started with a first denaturation step (95 oC for 5 min) followed by 35 repeats of temperature cycling steps (94 oC for 45 s, 55 oC for 45 s, and 72 oC for 90 s). The 1.2-kb PCR product was analyzed and gel-purified by agarose gel electrophoresis then mixed with vector pCR-XL-TOPO according to the TOPO® XL PCR Cloning Kit protocol (Invitrogen) to yield plasmid pTyrA, which was subjected to DNA sequence analysis. The tyrA ORF was excised from plasmid pTyrA by digestion with EcoRI and the 1.2-kb DNA fragment ligated to EcoRI-digested plasmid pJF tyrD maoA palR hpaBC (also referred to as plasmid pDMPH). Ligation mixtures were used to transform E. coli TOP10 competent cells. Ampicilling-resistant transformants were selected on LB solid medium and analyzed for tyrA insertion and correct orientation, which afforded plasmid pJF tyrA tyrD maoA palR hpaBC (also referred to as pTDMPH).

### EXAMPLES OF HYDROXYTYROSOL PRODUCTION FROM D-GLUCOSE

### Example 1

### Fermentative Production of Hydroxytyrosol from D-Glucose by E. coli TOP10/pDMPH Growing Cells

Inoculants were started by introducing 1 mL of E. coli TOP10/pDMPH from a working cell bank (frozen in 20% glycerol) into 5 mL of M9 inoculation medium containing the appropriate antibiotic, in this case ampicillin (100 mg/L). Cultures were grown for 24 h. An aliquot of this culture was transferred to 50 mL of M9 induction medium containing ampicillin (100 mg/L), to a starting OD₆₀₀ of 0.025-0.05 (1% inoculum). The 50 mL culture was grown at 37° C. with agitation at 250 rpm to OD600=0.5. Protein expression was then induced by addition of IPTG to a final concentration of 0.5 mM. The cultures were shaken at 37° C. and 250 rpm. Cell-free culture supernatants were analyzed by HPLC at several time-points in order to identify products and side-products formed. Typically, bacterial cultures were sampled just prior to IPTG addition to provide a background check (t=0); then 2-5 h after IPTG addition to detect potential biosynthetic intermediates; and finally 16-18 h after IPTG addition to measure product and side-product concentrations (see Table 3).

HPLC analysis of cell-free supernatants of cultures of E. coli TOP10/pDMPH show that no more than 0.2 mM L-tyrosine is consumed in the 17.5 h following IPTG induction, while over 0.8 mM hydroxytyrosol is produced by E. coli strain TOP10/pDMPH during this time. Therefore 0.6 mM of the hydroxytyrosol produced by E. coli strain TOP10/pDMPH growing in minimal medium must stem from D-glucose. E. coli strain TOP10/pDMPH, an E. coli K-12 derivative, can carry out the endogenous biosynthesis of L-tyrosine from D-glucose via the shikimate pathway and can produce hydroxytyrosol from L-tyrosine using plasmid-localized genes encoding L-tyrosine decarboxylase (tyrD), monoamine oxidase (maoA), phenylacetaldehyde reductase (palR) and 4-hydroxyphenylacetate 3-monooxygenase (hpaBC). This leads to the conclusion that hydroxytyrosol can be produced from a simple carbon source such as D-glucose by aerobic fermentation of a recombinant microorganism expressing an aromatic amino acid decarboxylase activity, an amine oxidase activity, an acetaldehyde reductase activity, and an aromatic hydroxylase activity and comprising the glycolysis pathway, the pentose phosphate pathway, and the aromatic amino acid biosynthesis pathway, or pathways derived therefrom.

TABLE 3
Evidence of hydroxytyrosol production from D-glucose
by E. coli TOP10/pDMPH growing cells.

| | | | Concentrations in culture medium (mM)^{c} | | |
|---|---|---|---|---|---|
| | Time | Biomass | L-Tyro- | Tyro- | Hydroxy- |
| Entry^{a} | (h)^{b} | (OD₆₀₀) | sine | sol | tyrosol |
| 1.0 | 0 | 0.4 | 0.55^{d} | 0 | 0 |
| 1.1 | 2.25 | 1.7 | 0.69 | 0.00 | 0.00 |
| 1.2 | 4.75 | 3.0 | 0.51 | 0.12 | 0.33 |
| 1.3 | 17.5 | 2.4 | 0.45 | 0.00 | 0.88 |
| 2.0 | 0 | 0.5 | 0.55^{d} | 0 | 0 |
| 2.1 | 2.25 | 2.1 | 0.65 | 0.09 | 0.05 |
| 2.2 | 4.75 | 2.7 | 0.50 | 0.15 | 0.43 |
| 2.3 | 17.5 | 3.7 | 0.33 | 0.00 | 0.84 |
| 3.0 | 0 | 0.6 | 0.55^{d} | 0 | 0 |
| 3.1 | 2.25 | 2.5 | 0.68 | 0.10 | 0.05 |
| 3.2 | 4.75 | 2.5 | 0.53 | 0.17 | 0.42 |
| 3.3 | 17.5 | 2.9 | 0.32 | 0.00 | 0.84 |

^{a}Entry series 1, 2 and 3 correspond to the above-described experiment executed in triplicate.
^{b}Time is counted starting from IPTG addition (t = 0).
^{c}As detected by HPLC analysis of cell-free culture supernatants.
^{d}Before IPTG addition tyrosine present in the culture medium from casamino acids.

### Example 2

### Production of Hydroxytyrosol from d-Glucose by E. coli TOP10/pDMPH Resting Cells

Inoculants were started by introducing 1 mL of E. coli TOP10/pDMPH from a working cell bank (frozen in 20% glycerol) into 5 mL of M9 inoculation medium containing the appropriate antibiotic (Ap, 100 mg/L). Cultures were grown for 24 h. An aliquot of this culture was transferred to 50 mL of M9 induction medium containing the appropriate antibiotic (Ap, 100 mg/L) to a starting OD₆₀₀ of 0.025-0.05 (1% inoculum). The 50 mL culture was grown at 37° C. with agitation at 250 rpm to OD600=0.5. Protein expression was then induced by addition of IPTG to a final concentration of 0.5 mM. The cultures were shaken at 37° C. and 250 rpm for 3 h. The cells were briefly chilled on ice, harvested by centrifugation (1800 g, 4° C., 10 min), then gently resuspended in 50 mL M9 medium supplemented with ampicillin (100 mg/L) and IPTG (0.5 mM), thus omitting addition of an external source of L-tyrosine such as casamino acids. Experiments were re-initiated by shaking cell suspensions at 37° C. and 250 rpm. Cell-free supernatants were analyzed by HPLC at several time-points in order to identify products and side-products formed. Typically, bacterial suspensions were sampled immediately after dispersing the cells in M9 medium for a background check and then at regular intervals in the course of the experiment (see Table 4). HPLC analyses of reaction supernatants free of E. coli TOP10/pDMPH cells show that 13.9-20.0 mg/L hydroxytyrosol are produced by E. coli strain TOP10/pDMPH directly from D-glucose. No other product or biosynthetic intermediate accumulated or were detected throughout the process. In the absence of exogenously added 1-tyrosine or other L-tyrosine-containing additives such as casamino acids, this experiment provides irrefutable proof that hydroxytyrosol is produced by E. coli TOP10/pDMPH cells from the only carbon source in the medium, namely D-glucose. Aerobic bioconversion of a simple carbon source such as D-glucose into hydroxytyrosol is possible using as biocatalyst a recombinant microorganism expressing an aromatic amino acid decarboxylase activity, an amine oxidase activity, an acetaldehyde reductase activity, and an aromatic hydroxylase activity and comprising the glycolysis pathway, the pentose phosphate pathway, and the aromatic amino acid biosynthesis pathway, or pathways derived therefrom.

TABLE 4
Evidence of hydroxytyrosol production from d-glucose
by E. coli TOP10/pDPMH resting cells

| | | | Concentrations in culture medium (mM)^{c} | | |
|---|---|---|---|---|---|
| | Time | Biomass | l-Tyro- | Tyro- | Hydroxy- |
| Entry^{a} | (h)^{b} | (OD₆₀₀) | sine^{d} | sol | tyrosol |
| 1.0 | 0 | 1.1 | 0 | 0 | 0 |
| 1.1 | 2.5 | 1.4 | 0 | 0 | 0 |
| 1.2 | 15 | 0.9 | 0 | 0 | 0.09 |
| 2.0 | 0 | 2.1 | 0 | 0 | 0 |
| 2.1 | 2.5 | 1.5 | 0 | 0 | 0 |
| 2.2 | 15 | 0.9 | 0 | 0 | 0.11 |
| 2.3 | 39 | 1.2 | 0 | 0 | 0.13 |
| 2.4 | 65 | 1.3 | 0 | 0 | 0.12 |

^{a}Entry series 1 and 2 correspond to duplicate runs of the experiment described above.
^{b}Time is counted starting from cells resuspension in M9 medium (t = 0).
^{c}As detected by HPLC analysis of cell-free culture supernatants.
^{d}Any L-tyrosine detected must stem from E. coli's endogenous biosynthesis pathway.

### Example 3

### Improved Hydroxytyrosol Biosynthesis from D-Glucose by E. coli TOP10/pTDMPH

Inoculants were started by introducing one single colony of E. coli TOP10/pTDMPH from a freshly streaked agar plate into 5 mL of M9 inoculation medium containing the appropriate antibiotic, in this case ampicillin (100 mg/L). Cultures were grown for 24 h. An aliquot of this culture was transferred to 50 mL of M9 induction medium containing ampicillin (100 mg/L), to a starting OD₆₀₀ of 0.025-0.05 (1% inoculum). The 50 mL culture was grown at 37° C. with agitation at 250 rpm to OD600=0.5. Protein expression was then induced by addition of IPTG to a final concentration of 0.5 mM. The cultures were shaken at 37° C. and 250 rpm. Cell-free culture supernatants were analyzed by HPLC at several time-points in order to identify products and side-products formed. Typically, bacterial cultures were sampled just prior to IPTG addition to provide a background check (t=0); then 3-4 h after IPTG addition to detect potential biosynthetic intermediates; and finally 19 h after IPTG addition to measure product and side-product concentrations (see Table 5).

HPLC analyses of cell-free culture supernatants show that in the control reaction with E. coli strain TOP10/pDMPH, no more than 0.1 mM 1-tyrosine is consumed in the 19 h following IPTG induction, while about 1.0 mM hydroxytyrosol and 0.3 mM tyrosol are produced during this time. Therefore 1.2 mM of D-glucose was funneled through the hydroxytyrosol biosynthetic pathway via 1-tyrosine by E. coli TOP10/pDMPH growing cells. In the case of E. coli strain TOP10/pTDMPH, which expresses the tyrA gene encoding chorismate mutase/prephenate dehydrogenase in addition to the genes encoding L-tyrosine decarboxylase (tyrD), monoamine oxidase (maoA), phenylacetaldehyde reductase (palR) and 4-hydroxyphenylacetate 3-monooxygenase (hpaBC), about 0.2 mM L-tyrosine is consumed in the 19 h following IPTG induction, while 2.0-2.4 mM hydroxytyrosol and 0-0.2 mM tyrosol are produced by E. coli strain TOP10/pDMPH during this time. Therefore 1.8-2.4 mM of D-glucose was engaged through the hydroxytyrosol biosynthetic pathway via L-tyrosine by E. coli TOP10/pTDMPH growing cells, amounting to a 1.5-2.0 fold increase as compared to E. coli TOP10/pDMPH.

This leads to the conclusion that increasing carbon flux through L-tyrosine biosynthesis by over-expression or up-regulation of chorismate mutase/prephenate dehydrogenase, or any other strategy well known to those skilled in the art, increases hydroxytyrosol production from a simple carbon source such as D-glucose by aerobic fermentation of a recombinant microorganism expressing an aromatic amino acid decarboxylase activity, an amine oxidase activity, an acetaldehyde reductase activity, and an aromatic hydroxylase activity and comprising the glycolysis pathway, the pentose phosphate pathway, and the aromatic amino acid biosynthesis pathway, or pathways derived therefrom.

TABLE 5
Increased hydroxytyrosol production from D-glucose
by E. coli TOP10/pTDPMH growing cells as compared
to E. coli TOP10/pDMPH growing cells.

| | | | Concentrations in culture medium (mM)^{c} | | |
|---|---|---|---|---|---|
| | Time | Biomass | L-Tyro- | Tyro- | Hydroxy- |
| Entry^{a} | (h)^{b} | (OD₆₀₀) | sine | sol | tyrosol |

Strain E. coli TOP10/pDMPH (control):

| | | | | | |
|---|---|---|---|---|---|
| 1.0 | 0 | 0.4 | 0.52^{d} | 0 | 0 |
| 1.1 | 3 | 1.7 | 0.64 | 0.13 | 0.09 |
| 1.2 | 4 | 3.0 | 0.60 | 0.20 | 0.23 |
| 1.3 | 19 | 2.4 | 0.41 | 0.32 | 0.99 |

Strain E. coli TOP10/pTDMPH:

| | | | | | |
|---|---|---|---|---|---|
| 2.0 | 0 | 0.5 | 0.53^{d} | 0 | 0 |
| 2.1 | 3 | 2.1 | 0.69 | 0.20 | 0.05 |
| 2.2 | 4 | 2.7 | 0.66 | 0.32 | 0.12 |
| 2.3 | 19 | 3.7 | 0.34 | 0 | 2.02 |
| 3.0 | 0 | 0.6 | 0.54^{d} | 0 | 0 |
| 3.1 | 3 | 2.5 | 0.67 | 0.22 | 0.07 |
| 3.2 | 4 | 2.5 | 0.63 | 0.34 | 0.15 |
| 3.3 | 19 | 2.9 | 0.35 | 0.22 | 2.38 |

^{a}Entry series 2 and 3 correspond to duplicate runs of the experiment described above.
^{b}Time is counted starting from IPTG addition (t = 0).
^{c}As detected by HPLC analysis of cell-free culture supernatants.
^{d}Before IPTG addition tyrosine present in the culture medium from casamino acids.

### Example 4

### Influence of D-Glucose Concentration on the Production of Hydroxytyrosol from L-Tyrosine and D-Glucose Using E. coli TOP10/pDMPH Growing Cells

The influence of glucose concentration on hydroxytyrosol production was evaluated. Shake-flask experiments were run in parallel, where E. coli strain TOP10/pDMPH was grown in M9 salts supplemented with casamino acids (20 g/L), MgSO₄ (1 mM), ampicillin (100 mg/L), and decreasing amounts of glucose (40-0.4 g/L). Cultivation and induction were carried out according to standard protocols. After a 3 h induction period, all shake-flasks were treated with the same amount of exogenous tyrosine to a total substrate concentration of ˜1.2 mM, ˜0.6 mM of which originate from casamino acids. Hydroxytyrosol production was monitored by HPLC. Results showed a noticeable trend with three categories: (i) shake-flasks with high glucose content (10-40 g/L) showed excellent hydroxytyrosol production from tyrosine and glucose as can be inferred from the 1.9-2.1 mM detected hydroxytyrosol by t=39 h; (ii) shake-flasks with medium glucose content (2.5-5 g/L) displayed a good hydroxytyrosol production reaching 1.4-1.6 mM by t=39 h; (iii) shake-flasks with a glucose content lower than 1 g/L were characterized by incomplete bioconversion of tyrosine into hydroxytyrosol with no more than 0.4-0.8 mM hydroxytyrosol detected at t=16 h followed by product decomposition as judged by the decrease in hydroxytyrosol titre to 0-0.5 mM at t=39 h. Glucose-rich cultivation conditions were shown to benefit hydroxytyrosol production by E. coli TOP10/pDMPH growing cells, therefore initial glucose concentration under standard conditions was set at 40 g/L.

### Example 5

### Influence of D-Glucose Concentration on the Production of Hydroxytyrosol from L-Tyrosine and D-Glucose Using E. coli TOP10/pDMPH Resting Cells

Similar experiments that evaluate the influence of glucose concentration on hydroxytyrosol production were designed using resting cells. E. coli strain TOP10/pDMPH was grown according to standard protocol, induced with IPTG and shaken for 3 h. Cells were harvested by centrifugation and resuspended in M9 salts supplemented with MgSO₄ (1 mM), IPTG (0.5 mM), and ampicillin (100 mg/L). Casamino acids were omitted from the medium to prohibit cellular growth. Cell suspensions were treated with tyrosine (˜1.0 mM) and glucose (0.4-40 g/L) and shaken at 37° C. Hydroxytyrosol production was analyzed by HPLC. Optical densities of all bacterial suspensions ranged between 1.8-2.1 before transfer and between 1.1-1.5 after transfer. Results could be sorted in two categories: (i) shake-flasks with higher glucose content (2.5-40 g/L) displayed almost equal titres of hydroxytyrosol (1.2±0.1 mM) and tyrosol (0.20±0.05 mM) from tyrosine and/or glucose by t=39 h; (ii) shake-flasks with a glucose content lower than 1 g/L were characterized by incomplete tyrosine-to-hydroxytyrosol bioconversion and formation of side-products. In the presence of 1 g/L glucose, hydroxytyrosol (0.6 mM), tyrosol (0.4 mM) 4-hydroxyphenylacetic acid (0.1 mM), and unreacted tyrosine (0.3 mM) were detected at t=39 h. In the presence of 0.4 g/L glucose, hydroxytyrosol (0.4 mM), tyrosol (0.1 mM), 4-hydroxyphenylacetic acid (0.1 mM), and unreacted tyrosine (0.4 mM) were detected at t=39 h. Glucose-rich conditions benefit to hydroxytyrosol production by E. coli TOP10/pDMPH resting cells.

### Example 6

### Influence of Copper(II) Ions on the Production of Hydroxytyrosol by E. coli TOP10/pDMPH Growing Cells

Two shake-flask experiments were run in parallel under standard cultivations, where E. coli strain TOP10/pDMPH was grown in M9 salts (50 mL) supplemented with glucose (40 g/L), casamino acids (20 g/L), MgSO₄ (1 mM), and ampicillin (100 mg/L). Cultures were grown at 37° C. with agitation at 250 rpm to OD₆₀₀=0.5. Gene expression was then induced by addition of IPTG to a final concentration of 0.5 mM. At this point, CuSO₄ was added to a final concentration of 50 μM to one culture; the other was left untreated. The cultures were shaken at 37° C. and 250 rpm for another 2-3 h. Experiments were initiated (t=0) by addition of ˜5.4 mM L-tyrosine. E. coli TOP10/pDMPH-catalyzed bioconversion of tyrosine (5.3 mM) was not complete in the absence of copper(II) resulting in no more than 60% mol/mol bioconversion: no residual tyrosine was detectable by HPLC analysis but only 3.2 mM hydroxytyrosol was produced within 18 h reaction time along with 2.8 mM tyramine and 0.1 mM tyrosol. In contrast, addition of 50 μM CuSO₄ to growing cultures of TOP10/pDMPH at the time of induction promoted excellent tyrosine-to-hydroxytyrosol bioconversion ratios. Up to 5.3 mM hydroxytyrosol was produced from 5.6 mM total starting substrates (5.4 mM tyrosine and 0.2 mM tyrosol) as detected by HPLC at t=0 h, resulting in a molar bioconversion ratio of 95% (mol/mol) in 18 h. Addition of copper(II) to growing E. coli TOP10/pDMPH cultures expressing hydroxytyrosol biosynthetic genes enhances the production of hydroxytyrosol from tyrosine and thus should benefit any process including or making use of tyrosine-to-hydroxytyrosol conversion.

1. A process for the fermentative production of Hydroxytyrosol (Hy-T), wherein a transformed host cell is cultivated under suitable culture conditions that allow the direct production of Hy-T from a carbon source obtainable from the D-glucose metabolization pathway and wherein the genome of said host cell is genetically engineered with a polynucleotide encoding an enzyme capable of transforming tyrosol to Hy-T and at least one polynucleotide encoding an enzyme which has an activity selected from the group consisting of:
phenylacetaldehyde reductase activity,
aromatic amino acid decarboxylase, for example L-phenylalanine and/or L-tyrosine decarboxylase activity,
monoamine oxidase activity,
a lyase activity,
phenylpyruvate decarboxylase activity,
monophenol monooxygenase, for example a tyrosinase activity,
toluene monooxygenase, for example, toluene para-monooxygenase activity,
phenylalanine-4-hydroxylase and/or pterin-4-alpha-carbinolamine dehydratase activity, and
chorismate mutase and/or perphenate dehydrogenase activity.

2. The process according to claim 1, wherein the polynucleotide encoding an enzyme capable of transforming tyrosol to Hy-T is selected from the group consisting of
a) polynucleotides encoding a protein comprising the amino acid sequence according to SEQ ID NO: 2, SEQ ID NO: 6, SEQ ID NO: 8, SEQ ID NO: 39, or SEQ ID NO: 41;
b) polynucleotides comprising the nucleotide sequence according to SEQ ID NO: 1, SEQ ID NO: 5, SEQ ID NO: 7, SEQ ID NO: 38, or SEQ ID NO: 40;
c) polynucleotides encoding a fragment or derivative of a polypeptide encoded by a polynucleotide of any of (a) or (b) wherein in said derivative one or more amino acid residues are conservatively substituted compared to said polypeptide;
d) polynucleotides the complementary strand of which hybridizes under stringent conditions to a polynucleotide as defined in any one of (a) to (c);
e) polynucleotides which are at least 90 or 95% homologous to a polynucleotide as defined in any one of (a) to (d); and
f) complementary strands of a polynucleotide as defined in (a) to (e).

3. The process according to claim 1, wherein the at least one additional polynucleotide encoding an enzyme which has an activity selected from the group consisting of phenylacetaldehyde reductase activity, L-phenylalanine and/or L-tyrosine decarboxylase activity, monoamine oxidase activity, a lyase activity, phenylpyruvate decarboxylase activity, toluene monooxygenase, for example, toluene para-monooxygenase activity, phenylalanine-4-hydroxylase and/or pterin-4-alpha-carbinolamine dehydratase activity, and chorismate mutase and/or perphenate dehydrogenase activity, is selected from the group consisting of:
a) polynucleotides encoding a protein comprising the amino acid sequence according to SEQ ID NO: 4, SEQ ID NO: 10, SEQ ID NO: 12, SEQ ID NO: 14, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, or SEQ ID NO: 43;
b) polynucleotides comprising the nucleotide sequence according to, SEQ ID NO: 3, SEQ ID NO: 9, SEQ ID NO: 11 and SEQ ID NO: 13, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32. SEQ ID NO: 34, SEQ ID NO: 36, SEQ ID NO: 42;
c) polynucleotides encoding a fragment or derivative of a polypeptide encoded by a polynucleotide of any of (a) or (b) wherein in said derivative one or more amino acid residues are conservatively substituted compared to said polypeptide;
d) polynucleotides the complementary strand of which hybridizes under stringent conditions to a polynucleotide as defined in any one of (a) to (c);
e) polynucleotides which are at least 90 or 95% homologous to a polynucleotide as defined in any one of (a) to (d); and
f) complementary strands of a polynucleotide as defined in (a) to (e).

4. The process according to claim 1, wherein the non-transformed wild type of said host cell is capable of producing either L-tyrosine, L-phenylalanine, phenylpyruvate or hydroxyphenylpyruvate from glucose.

5. The process according to any claim 1 4, characterized in that glutathione and/or glycerol and/or ascorbic acid is added to the reaction medium.

6. The process according to claim 1, characterized in that a copper(II) salt is added to the reaction medium.

7. The process according to claim 1, wherein hydroxytyrosol is produced by resting cells.

8. The process according to claim 1, wherein hydroxytyrosol is produced by growing cells.

9. A genetically engineered host cell able to produce hydroxytyrosol from a carbon source obtainable from the D-glucose metabolization pathway, wherein said host cell is genetically engineered with a polynucleotide encoding an enzyme capable of transforming tyrosol to Hy-T and at least one polynucleotide encoding an enzyme which has an activity selected from the group consisting of:
phenylacetaldehyde reductase activity,
aromatic amino acid decarboxylase, for example L-phenylalanine and/or L-tyrosine decarboxylase activity,
monoamine oxidase activity,
a lyase activity,
phenylpyruvate decarboxylase activity,
monophenol monooxygenase, for example a tyrosinase activity,
toluene monooxygenase, for example, toluene para-monooxygenase activity,
phenylalanine-4-hydroxylase and/or pterin-4-alpha-carbinolamine dehydratase activity, and
chorismate mutase and/or perphenate dehydrogenase activity.

10. The microorganism according to claim 9, which has been transformed or transfected by at least one polynucleotide selected from the group consisting of:
a) polynucleotides encoding a protein comprising the amino acid sequence according to SEQ ID NO: 2 SEQ ID NO: 4 SEQ ID NO: 6, SEQ ID NO: 8 SEQ ID NO: 10, SEQ ID NO: 12 SEQ ID NO: 14 SEQ ID NO: 17 SEQ ID NO: 19 SEQ ID NO: 21, SEQ ID NO: 23, SEQ ID NO: 25, SEQ NO: 27, SEQ ID NO: 29 SEQ ID NO: 31 SEQ ID NO: 33 SEQ ID NO: 35 SEQ ID NO: 37. SEQ ID NO; 39, SEQ ID NO: 41, or SEQ ID NO: 43;
b) nucleotides comprising the nucleotide sequence according to SEQ ID NO: 1 SEQ ID NO: 3, SEQ ID NO: 5, SEQ ID NO: 7, SEQ ID NO: 9, SEQ ID NO: 38, SEQ ID NO: 11 and SEQ ID NO: 13. SEQ ID NO: 16 SEQ ID NO: 18. SEQ ID NO: 20, SEQ ID NO: 22 SEQ ID NO: 24 SEQ ID NO: 26 SEQ ID NO: 28 SEQ ID NO: 30, SEQ ID NO: 32, SEQ ID NO: 34, SEQ ID NO: 36, SEQ ID NO: 40, or SEQ ID NO: 42;
c) polynucleotides encoding a fragment or derivative of a polypeptide encoded by a polynucleotide of any of (a) or (b) wherein in said derivative one or more amino acid residues are conservatively substituted compared to said polypeptide;
d) polynucleotides the complementary strand of which hybridizes under stringent conditions to a polynucleotide as defined in any one of (a) to (c);
e) polynucleotides which are at least 90 or 95% homologous to a polynucleotide as defined in an one of a to d and
f) complementary strands of a polynucleotide as defined in (a) to (e).

11. The microorganism according to claim 9, which is engineered to comprise a nucleotide sequence selected from the group consisting of
a) nucleotide sequences encoding a protein comprising the amino acid sequence according to SEQ ID NO: 4 and SEQ ID NO: 6 and SEQ ID NO: 8 and SEQ ID NO: 12 respectively and
b) nucleotide sequences according to SEQ ID NO: 3 and SEQ ID NO: 5 and SEQ ID NO: 7 and SEQ ID NO: 11.

12. The microorganism according to claim 9, which is engineered to comprise a nucleotide sequence selected from the group consisting of:
c) nucleotide sequences encoding a protein comprising the amino acid sequence according to SEQ ID NO: 4 and SEQ ID NO: 6 and SEQ ID NO: 8 and SEQ ID NO: 12 and SEQ ID NO: 14 respectively and
d) nucleotide sequences according to SEQ ID NO: 3 and SEQ ID NO: 5 and SEQ ID NO: 7 and SEQ ID NO: 11 and SEQ ID NO: 13.

13. The A microorganism according to claim 9, which is engineered to comprise a nucleotide sequence selected from the group consisting of:
e) nucleotide sequences encoding a protein comprising the amino acid sequence according to SEQ ID NO: 4 and SEQ ID NO: 6 and SEQ ID NO: 8 and SEQ ID NO: 12 and SEQ ID NO: 14 and SEQ ID NO: 43 respectively and
f) nucleotide sequences according to SEQ ID NO: 3 and SEQ ID NO: 5 and SEQ ID NO: 7, SEQ ID NO: 11 and SEQ ID NO: 13 and SEQ ID NO: 42.
